(19) United States
(12) Patent Application Publication
(10) Pub. No.: US 2022/0168442 A1
(43) Pub. Date: Jun. 2, 2022

(54) ARTIFICIAL T-CELL STIMULATING MATRIX FOR IMMUNOTHERAPY

(71) Applicant: The Johns Hopkins University, Baltimore, MD (US)

(72) Inventors: Jonathan Schneck, Baltimore, MD (US); Hai-Quan Mao, Balitmore, MD (US); John Wirthlin Hickey, Balitmore, MD (US)

(21) Appl. No.: 17/600,840

(22) PCT Filed: Apr. 2, 2020
(86) PCT No.: PCT/US2020/026395

§ 371 Date: Oct. 1, 2021

Related U.S. Application Data
(60) Provisional application No. 62/828,087, filed on Apr. 2, 2019.

(51) Int. Cl. A61K 47/69; A61K 47/68; C07K 16/28; C12N 5/0783; A61K 35/17; A61P 35/00

(52) U.S. Cl. CPC A61K 47/6903; A61K 47/6849; C07K 16/2809; C12N 2533/80; C12N 5/0636; A61K 35/17; A61P 35/00; C07K 16/2818

(57) ABSTRACT

Provided herein is a composition comprising a hydrogel having a shear modulus of about 20 Pa to about 1600 Pa conjugated with anti-CD3 antibodies and anti-CD28 antibodies. Also provided are methods of activating T cells and methods of killing cancer cells using the hydrogel composition.

### BACKGROUND OF THE INVENTION

With the success of checkpoint blockade, adoptive cell transfer (ACT), and chimeric antigen receptor (CAR) T cell therapy, T lymphocytes (also known as “T cells”) are increasingly targeted and utilized in immunotherapies (Hickey et al., in Biology of T Cells—Part A (eds. Galluzzi, & Rudqvist) 341: 277-362 (Academic Press, 2018). For both ACT and CAR T cell therapies, T cells must be removed from patients, cultured and stimulated ex vivo, and then reinjected into patients for cancer immunotherapy (Rosenberg et al., Nat. Rev. Cancer, 8: 299 (2008)). This presents two major challenges. First, the number of T cells needed is very large, requiring culturing for 6-8 weeks at a time, resulting in potential loss or alteration of the cells' functionality and phenotype to mediate effective killing and long-term memory (Ahmadzadeh et al., Blood, 114: 1537-1544 (2009); Pollack et al., J Immunother. Cancer, 2: 36 (2014); Wherry, E. J., Nat. Immunol., 131: 492-499 (2011); and Wherry et al., Nat. Immunol., 4: 225 (2003)). Second, antigen-specific stimulations utilize antigen-presenting cells (APCs) that may be immunosuppressed and are often dysfunctional, or non-specific stimulation from synthetic surfaces through CD3 can result in expansion of irrelevant and potentially harmful clones (Della Bella et al., Br. J. Cancer, 89: 1463 (2003); Satthaporn et al., Cancer Immunol. Immunother., 53: 510-8 (2004); and Ye et al., J. Exp. Clin. Cancer Res., 29: 78 (2010)). By improving the quality or phenotype and functionality ex vivo, therapeutic outcomes can be improved significantly (Hinrichs et al., Blood, 117(3): 808-14 (2011)).

Approaches for addressing these challenges include altering the composition of cytokine cocktails, signaling pathway inhibitors, and feeder cells (Rosenberg, S. A. & Restifo, N. P., Science, 348: 62-68 (2015)). Additionally, the two signals necessary to stimulate the T cell receptor and co-stimulatory molecules have been conjugated to synthetic materials: inorganic or polymeric particles (Hickey et al., Nano Lett., 17, (2017); Oelke et al., Nat. Med., 9: 619-24 (2003); and Fadel et al., Nat. Nanotechnol., 9: 639-647 (2014); Kosmides et al., Biomaterials, 118: 16-26 (2017) and surfaces (Cheung et al., Nat. Biotechnol., 36: 160-169 (2018); Judokusumo et al., Biophys. J., 102: L5-L7 (2012); and O'Connor et al., J. Immunol., 189: 1330-1339 (2012)). Current synthetic T cell stimulation platforms are helpful in efficiently enriching and activating antigen-specific T cells (Kosmides et al., Nano Lett., 18 (2018); Perica et al., ACS Nano, 9: 6861-6871 (2015); and Hickey et al., Biomaterials (2018)), providing cell membrane-mimetic materials (Cheung et al., supra), and acting as in vitro or in vivo stimulators (Kosimedes et al., supra); however, none provide environmental cues similar to what T cells encounter in the lymphoid organs, such as the spleen or lymph node.

The extracellular matrix (ECM) is an important regulator of cellular function, including gene expression, differentiation, migration, proliferation, and morphology (Adams, J. C. & Watt, F. M., Nature, 340: 307 (1989); Watt, F. M., Curr. Opin. Cell Biol., 1: 1107-1115 (1989); Lynch et al., Exp. Cell Res., 216: 35-45 (1995); and Bissell, M. J. and Barcellos-Hoff, M. H., J Cell Sci, 8: 327-343 (1987)). T lymphocytes primarily reside in the lymphoid organs. These unique microenvironments enable rapid communication, cell differentiation, and allow antigen-specific cells to expand thousands-fold in response to infection (Gretz et al., Immunol. Rev., 156: 11-24 (1997); Ayroldi et al., Blood, 86: 2672-2678 (1995); Pozzi et al., J Cell Biol., 142: 587-594 (1998); and Petrie, H. T., Nat. Rev. Immunol., 3: 859 (2003)). Although it is well known that cells are influenced by ECM properties such as composition, stiffness, and bioactive cues that create unique microenvironments suited to the function of each cell and tissue (Engler et al., Cell, 126: 677-689 (2006)), the role of ECM on T cell activation has not been investigated.

There remains a need for compositions and methods that can improve the functionality and phenotype of T cells stimulated ex vivo for therapeutic applications.

### BRIEF SUMMARY OF THE INVENTION

The disclosure provides a composition comprising a hydrogel conjugated to a first molecule that provides a first T cell activating signal and a second molecule that provides a second T cell activating signal. The disclosure also provides methods of activating T cells and methods of killing cancer cells using the aforementioned composition.

### BRIEF DESCRIPTION OF THE DRAWING(S)

FIG. 1 is a schematic showing the three main components and conjugation chemistry of the hyaluronic acid hydrogels to form the artificial T cell stimulating matrix (aTM).

FIG. 2A is a schematic of aTM made from conjugating Signals 1 and 2 to a hyaluronic acid hydrogel. Attachment of Signal 1 and 2 enable effective T cell stimulation which leads to T cell proliferation, differentiation, and effector function. Receptors bind to ECM hydrogel and also contribute to attachment and T cell signaling. FIGS. 2B-2E are graphs illustrating B6 CD8+ T cell fold expansion measured after seven days of stimulation of the antigen-specific T cells on the hydrogels with (B) Signals 1+2 conjugated or soluble (error bars show s.e.m.; **p<0.005, n=4, Student's t-test, two-tailed), (C) conjugated together or alone (error bars show s.e.m.; **p<0.005, ***p<0.0005, n=5-7, one-way ANOVA with Tukey's post test), and (D) at varying amounts of Signals 1+2, n=5. (E) Day 7 CD8+ T cell fold expansion measured after seven days of stimulation of the antigen-specific T cells on the aTM. T cells were removed from aTM on the day noted and cultured on TCP until day 7.

FIG. 3A is a graph illustrating conjugation efficiency and linear estimated ligand spacing on aTM hydrogels. Fluorescent antibody levels in the hydrogel washes were below level of detection, and show efficient conjugation efficiency (error bars show s.e.m., n=5). FIG. 3B is a graph showing estimated ligand spacing based on the Signal 1 and 2 concentration in the aTM solution.

FIG. 4 is a graph illustrating the viability of stimulated T cells measured on day 7 by trypan blue staining and hemocytometry for varying amounts of Signals 1+2 conjugated to hydrogels, n=4-5.

FIG. 5A is a schematic illustrating hypothesis that tuning stiffness of aTM will change the ability for mechanotransduction of TCR signaling. FIG. 5B is a graph showing elastic modulus measured by rheometry with varying PEGDA crosslinker weight percent (error bars show s.e.m., n=3). FIG. 5C is a graph showing CFSE proliferation dye dilution measured after 3 days of stimulation of T cells comparing a stiff (3 kPa) and soft (0.5 kPa) aTM. FIG. 5D is a graph showing CD8+ T cell fold expansion measured after seven days of stimulation of the T cells on aTMs with varying stiffness (error bars show s.e.m.; *p<0.05, **p<0.005, n=4-12, one-way ANOVA with Tukey's post test). FIG. 5E is a graph showing CD8+ T cell fold expansion measured for T cells stimulated on soft aTMs (0.5 kPa) with or without blebbistatin (error bars show s.e.m.; ***p<0.0005, n=4, Student's t-test, two-tailed). FIG. 5F is a graph showing quantitation of percentage of T cells in each divisional generation based on CFSE proliferation dye dilution with T cells stimulated on HA hydrogels of different stiffness with aAPC (error bars show s.e.m, n=4-8). (G) CD8+ T cell fold expansion measured after seven days of stimulation of T cells on the aTMs with either laminin and RGD attached (error bars show s.e.m.; *p<0.05, n=3-6, one-way ANOVA with Tukey's post test).

FIG. 6 is a graph illustrating CD8+ T cell fold expansion measured after seven days of stimulation of the T cells on aTMs with varying stiffness (error bars show s.e.m.; *p <0.05, **p<0.005, n=4-12, one-way ANOVA with Tukey's post test).

FIG. 7 is a schematic of artificial antigen-presenting cell (aAPC) with Signal 1 (pMHC) and Signal 2 (aCD28) attached to a particle platform.

FIG. 8A is a graph showing CD8+ T cell fold expansion from T cells stimulated by aAPC on different hydrogel stiffness (0.5 kPa, 3 kPa) (error bars show s.e.m., n=14-15). FIG. 8B is a graph showing phenotypic markers (CD62L, CD44) measured by flow cytometry after 7 days of stimulation an (error bars show s.e.m.; n=18, Student's t-test).

FIGS. 9A and 9B are images showing that T cells attach to aTMs when Signals 1 and 2 are conjugated and are soft (0.5 kPa). Light video microscopy over a period of 24 hours was done to track cell movement and attachment to the aTMs of (FIG. 9A) 0.5 kPa (FIG. 9B) 3 kPa both with and without Signals 1 and 2 at time points of t=0 and t=24 hr.

FIG. 10 is a series of light microscopy images of T cell cultures on aTMs at day 3 with different stiffness (0.5, 3 kPa) and proteins (cyclic RGD, laminin) attached (scale bar=1 mm).

FIG. 11A is a schematic showing experimental setup testing the difference between activating antigen-specific CD8+ T cells with nanoparticle artificial antigens presenting cells (aAPC) on HA hydrogel versus a tissue culture plate (TCP). FIG. 11B is a graph showing CFSE proliferation dye dilution measured after 3 days of stimulation of antigen-specific T cells stimulated by the same dose of aAPC on either TCP or on HA hydrogel surface. FIG. 11C is a graph showing the percent of CD8+ T cells that have divided by day 3 as measured by CFSE proliferation dye dilution (error bars show s.e.m., *p<0.05, **p<0.005, ***p<0.0005 n=7, one-way ANOVA with Tukey's post test). FIG. 11D is a graph showing a time course experiment using p-S6 (S240/S244) as the read out for mTORC1 activation. This relative fold-change pattern represents three independent experiments using phospho-flow cytometry. FIG. 11E is a graph showing phenotypic markers (CD62L, CD44) measured by flow cytometry after 7 days of stimulation with aAPC on different surfaces (error bars show s.e.m.; *p<0.05, n=7, Student's t-test, two-tailed). FIGS. 11F and 11G show a time course experiment detecting fold change of (11F) IL15Ra (CD215) and (11G) IL7Ra (CD127). Geometric means of each data point are compared first with their isotype controls followed by the baseline control. Data represents two independent experiments. FIG. 11H is a graph showing T cells positive for all four cytokine and functional molecules (IL-2, IFN-γ, TNFα, CD107a) were measured by flow cytometry after 7 days of stimulation (error bars show s.e.m.; *p<0.05, n=7, Paired t-test, two-tailed).

FIG. 12 is a graph illustrating quantitation of percentage of T cells in each divisional generation based on CFSE proliferation dye dilution for the experiment described in FIG. 3B (error bars show s.e.m.; *p<0.05, **p<0.005, ***p<0.0005, n=5, Student's t-test).

FIG. 13A is a graph showing CFSE proliferation dye dilution measured after 3 days of culture of T cells on either tissue culture plates (TCPs) or on hyaluronic acid hydrogels (HA) without stimulatory signals present. FIG. 3B is a graph showing quantitation of percentage of T cells in each divisional generation based on CFSE proliferation dye dilution (error bars show s.e.m., n=15). FIG. 13C is a graph showing CD8+ T cell fold proliferation (error bars show s.e.m.; *p<0.05, n=7, Student's t-test) and FIG. 13D is a graph showing CD8+ T cell viability (error bars show s.e.m., n=8-11) measured after seven days of stimulation of the antigen-specific T cells on the hydrogels with or without aAPC.

FIG. 14 is a series of images illustrating time course experiment for T cell activation on HA surfaces compared to TCP surfaces for mTOR signaling. Cells were collected for western blot at designated time points after activation. Read out for mTORC1: p-S6K1 (T389) and p-S6 (S240/244); read out for mTORC2: p-AKT (S473). These images represent five independent experiments.

FIG. 15A is an image representing three independent experiments showing CD8+ T cells were treated with different drug conditions. Erki=U0126, an inhibitor that specifically targets Erk1/2; Rapa=Rapamycin, an inhibitor that specifically targets mTORC1. Image represents three independent experiments. FIG. 15B is a graph illustrating quantified data from western blot representing levels of expression of mTORC1 24 hours after activation, using p-S6 (S240/244) as a readout. The fold change pattern represents three independent experiments.

FIG. 16A is a graph showing CD8+ T cell fold expansion measured after seven days of stimulation by aTM with Signals 1+2 (anti-CD3 and anti-CD28) conjugated at varying amounts, n=3 independent donors. FIG. 16B is a graph showing the phenotype of CD8+ T cells after culture on aTM surfaces of varying Signals 1+2 amounts defined by CD45RA and CD62L (error bars show s.e.m). FIG. 16C is a graph showing CFSE proliferation dye dilution measured after 3 days of stimulation of CD8+ T cells comparing a stiff (3 kPa) and soft (0.5 kPa) aTM, n=3 independent donors. FIG. 16D is a graph showing CD8+ T cell fold expansion measured after seven days of stimulation on aTMs with varying stiffness (error bars show s.e.m.; **p<0.01, n=3 independent donors, one-way ANOVA with Dunnett's post test comparing to 3 kPa condition). FIG. 16E is a graph showing the phenotype of CD8+ T cells after culture on aTM surfaces of varying stiffness defined by CD45RA and CD62L (error bars show s.e.m) n=3 independent donors.

FIG. 17 is a graph showing quantitation of percentage of human CD8+ T cells in each divisional generation based on CFSE proliferation dye dilution with T cells stimulated on aTM with different density of anti-human CD3/CD28 (error bars show s.e.m, n=3 independent donors).

FIG. 18 is a series of light microscopy images of T cell cultures on aTMs with different density of aCD3/CD28 attached (0, 0.1, 1, 4, 10, and 25 μg/mL) (scale bar=1 mm).

FIG. 19 is a graph showing quantitation of percentage of human CD8+ T cells in each divisional generation based on CFSE proliferation dye dilution with T cells stimulated on aTM with different stiffness with 4 μg/mL anti-CD3/CD28 (error bars show s.e.m, n=3 independent donors).

FIG. 20 is a series of light microscopy images of T cell cultures on aTMs with different stiffness (0.5, 1, 1.7, 2.5, 3 kPa) (scale bar=1 mm).

FIG. 21A is a graph showing the percentage of antigen-specific T cells after 7 days of stimulation is determined by staining with cognate and non-cognate antigen-loaded peptide major histocompatibility complex (pMHC) and anti-CD8a. FIGS. 21B and 21B are graphs showing percentages (21B) and numbers (21C) of antigen-specific CD8+ T cells stimulated by aTM, or by aAPC on either TCP or HA hydrogel surface (error bars show s.e.m.; **p<0.01, ***p<0.001, n=12-15, one-way ANOVA with Tukey's post test). FIG. 21D is a graph showing fold IL7Ra expression on antigen-specific CD8+ T cells from HA+aAPC and aTM compared to IL7Ra expression on antigen-specific CD8+ T cells from TCP+aAPC (error bars show s.e.m., n=8-9). FIG. 21E is a series of pie charts showing T cell functionality measured by the number of functional molecules co-expressed by each antigen-specific cell (IFN-γ, TNFα, CD107a) after 7 days of stimulation (n=5-7). FIG. 21F is a schematic showing a murine melanoma therapeutic in vivo model for adoptively transferred cells. FIG. 21G is a graph showing tumor size measurements indicating that adoptive T cells from aTM stimulation significantly delayed tumor growth. Significance measured by two-way ANOVA with Bonferroni post-test (p<0.0001). FIG. 21G is a graph showing that that adoptive T cells from aTM stimulation significantly extended survival. Significance measured by log-rank test (p=0.05, n=5-6).

FIGS. 22A-22C are graphs illustrating intracellular cytokine and functionality staining of antigen-specific CD8+ T cells on day 7 reveals that aTM stimulations provide functional cells by CD107a (22A), IFNγ (22B), TNFα (22C) staining (n=5-7, error bars represent s.e.m.).

FIG. 23A is a schematic illustrating experimental set up to test whether aTM can provide additional support to stimulate tumor-experienced antigen-specific T cells. FIG. 23B is a graph showing the number of antigen-specific CD8+ T cells at day 7 of culture as measured by hemocytometry and cognate dimer staining by flow cytometry (error bars show s.e.m.; *p<0.05, **p<0.01, n=5, one-way ANOVA with Tukey's post test). FIG. 23C is a graph showing the size of B16-SIY tumors at day 12 post-injection (n=4). FIG. 23D is a graph showing the percent of antigen-specific CD8+ T cells at day 7 of culture as measured by cognate dimer staining by flow cytometry (error bars show s.e.m.; **p<0.01, n=5, one-way ANOVA with Tukey's post test).

FIG. 24A contain two graphs showing aTM-stimulated CMV+, CD8+ T cells stained on day 7 of culture with cognate CMV-tetramer and non-cognate Ml-tetramer to determine the percentage of antigen-specific T cells. FIG. 24B is a graph showing that aTM produce more than four times as many CMV+, CD8+ T cells by day 7 than controls: peptide pulsing without Signal 1 and 2 attached or no-peptide with Signal 1 and 2 attached (n=1).

FIGS. 25A-D are a series of graphs showing individual growth curves from mice with tumors for each treatment group (A) no treatment, (B) TCP+aAPC, (C) HA+aAPC, and (D) aTM.

FIG. 26A is a graph showing fold expansion of CD8+ T cells at day 7. * =p-value <0.05. FIG. 26B is a graph showing phenotypic characterization of expanded cells from the different aLNs of the 570-Pa stiffness. FIG. 26C is a graph showing CFSE staining of 2C CD8+ T cells from tail bleeds from either PBS or aLN treated B6 mice.

FIG. 27 is a graph showing percent killing by in vivo, aLN-activated antigen-specific T cells.

FIG. 28A is a graph showing stimulation on nanofibers with anti-CD3 and anti-CD28 conjugated to the surface mediates effective T cell activation. FIGS. 28B-28F are a series of graphs showing that composite aLN stimulates effective T cell proliferation as indicated by day 7 fold proliferation (28B), CFSE dilution analysis on day 3 (28C), CD8+ T cell phenotype on day 7 (28D), and intracellular cytokine staining indicative of cell function on day 7 (28E and 28F).

FIG. 29A is an image of microgel particles having a size close to 150 μm. FIG. 29B. is a graph showing that the microgel particles stimulate CD8+ T cells effectively by CFSE dilution when cultured in a bioreactor to enhance CD8+ T cell interaction. FIG. 29C is a graph showing effective T cell activation by nanofiber composite hydrogel aTM materials.

FIG. 30 is a pair of graphs showing percent killing from in vivo activated naive T cells with aTM.

FIG. 31 is a graph demonstrating that CD4+ T cells show differential amounts of proliferation on a range of aTM shear moduli. A series of CD4+ T cell-specific NHC aTM matrices was prepared with a shear moduli of between about 0.5 kPa to about 3.5 kPa and showed that CD4+ T cells were sensitive to aTM stiffness with the proliferation peaked at 1 kPa.

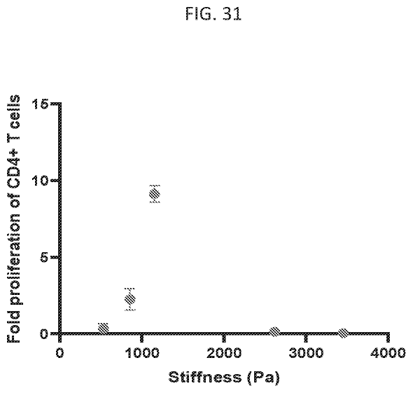

FIG. 32 shows the ratio of Th1 to Treg cells after plating naïve CD4+ T cells on the aTM either with skewing cytokines incorporated or with general cytokine media (TF media). The cytokine milieu is known to affect CD4+ lineage determination, which has a marked effect on therapy outcomes. For example, Th1 CD4+ T cells are known to be beneficial for cancer applications. Here, naïve CD4+ T cells were plated and their specific differentiation was induced through the incorporation of Th1-skewing cytokines (IL-2, IL-12, IFNγ) into the aTM.

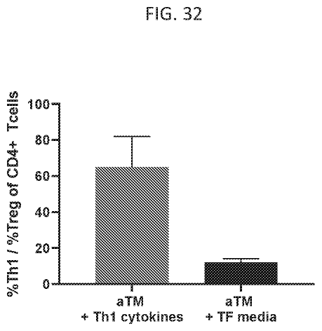

### DETAILED DESCRIPTION OF THE INVENTION

The disclosure is predicated, at least in part, on the discovery that an engineered artificial T cell stimulating matrix (aTM) mimics the critical features of both the natural extracellular matrix (ECM) and antigen presenting cells. The aTM described herein represents the first ECM-based T cell activation biomaterial which provides an optimal ex vivo environment for T cell activation. The aTM described herein also has the potential to be applied for direct T cell activation in vivo, eliminating the need for ex vivo T cell manipulation used in standard adoptive cell transfer (ACT) procedures.

The present disclosure provides a composition comprising a hydrogel having a shear modulus of about 20 Pa to about 1600 Pa, including about 20, 30, 40, 50, 60, 70, 80, 90, 100, 200, 300, 400, 500, 600, 700, 800, 900, 1000, 1100, 1200, 1300, 1400, 1500, and 1600 Pa. The term “hydrogel,” as used herein, refers to a three-dimensional network composed of hydrophilic polymers crosslinked either through covalent bonds or via physical intramolecular or intermolecular interactions. Hydrogels can absorb large amounts of water or biological fluids (up to several thousand percent), and swell readily without dissolving. The high hydrophilicity of hydrogels is primarily due to the presence of hydrophilic moieties such as carboxyl, amide, amino, and hydroxyl groups distributed along the backbone of polymeric chains. In the swollen state, hydrogels are soft and rubbery, closely resembling living tissues. Many hydrogels, such as chitosan and alginate-based hydrogels, exhibit desirable biocompatibility (see, e.g., El-Sherbiny, I. M., and Yacoub, M. H. Global Cardiology Science & Practice, 2013(3): 316-342 (2013); and Kyung et al, J. Appl. Polym. Sci., 83: 128-136 (2002)). Since their discovery more than 50 years ago, hydrogels have been employed in a variety of applications including, for example, drug delivery, wound healing, ophthalmic materials, and tissue engineering (see, e.g., El-Serbiny and Yacoub, supra; Hoffman, A. S., Ann. NY Acad Sci., 944: 62-73 (2001); and Peppas et al., Eur. J. Pharm. Biopharm., 50: 27-46 (2000)).

Hydrogels typically reach their equilibrium swelling when a balance occurs between osmotic driving forces, which encourage the entrance of water or biological fluids into the hydrophilic hydrogel matrix, and the cohesive forces exerted by the polymer strands within the hydrogel. These cohesive forces resist the hydrogel expansion and the extent of these forces depends particularly on the hydrogel crosslinking density. Generally, the more hydrophilic the polymer forming the hydrogel, the higher the total water amount absorbed by the hydrogel. Likewise, the higher the crosslinking extent of a particular hydrogel, the lower the extent of the gel swelling. Hydrogels in their dried forms are referred to in the art as “xerogels,” while dry porous hydrogels resulting from the use of drying techniques (e.g., freeze-drying or solvent extraction) are referred to in the art as “aerogels” (see, e.g., Guenet, J. M., Thermoreversible gelation of polymers and biopolymers; Academic Press, New York (1992), p. 89).

Hydrogels can be classified based on a variety of characteristics, such as, for example origin, durability, response to stimuli, charge, structure, and composition. With respect to origin, hydrogels can be classified as natural, synthetic or semi-synthetic. Most synthetic hydrogels are synthesized by traditional polymerization of vinyl or vinyl-activated monomers. The equilibrium swelling values of these synthetic hydrogels vary widely according to the hydrophilicity of the monomers and the crosslinking density. Natural hydrogels typically are made of natural polymers including, for example, polynucleotides, polypeptides, and polysaccharides that can be obtained from a variety of sources (e.g., collagen from mammals and chitosan from shellfish exoskeletons). With respect to durability, hydrogels can be classified as durable (such as most polyacrylate-based hydrogels) or biodegradable (such as polysaccharide-based hydrogels), depending on their stability characteristics in a physiological environment. Biodegradable hydrogels have recently been developed in which degradable polymers inside the hydrogel matrices undergo chain scission to form oligomers of low molecular weight. The resulting oligomers are either eliminated by the organism or undergo further degradation. Such biodegradable hydrogels can be used in both biomedical and non-biomedical applications (see e.g., Zhu, W. and Ding, J., J. Appl Polym Sci., 99: 2375 (2006)). With respect to response to environmental stimuli, “smart” hydrogels have been developed that exhibit changes in swelling behavior, network structure, and/or mechanical characteristics in response to various environmental stimuli such as pH, temperature, light, ionic strength or electric field (see, e.g., Gutowska et al., J Control Release, 22: 95-104 (1992); Ferreira et al, Int J Pharm., 794: 169-180 (2000); and D'Emanuele, A. and Staniforth, J. N., Pharm Res., 8: 913-918 (1991)). These changes typically disappear upon removal of the stimulus and the hydrogels are restored to their original state in a reversible manner.

Hydrogels can be used in a variety of tissue engineering applications, such as, for example, carriers for cell transplantations, scaffolds, barriers against restenosis, and drug depots. In one embodiment, the hydrogel can form a scaffold. The term “scaffold” refers to a structure that provides a platform for cell function, adhesion, and transplantation. Hydrogel scaffolds typically are used to provide bulk and mechanical structures to a tissue construct, whether cells are suspended within or adhered to a three-dimensional hydrogel framework. When a cellular-hydrogel adhesion is preferred over a suspension within the scaffold, inclusion of appropriate peptide moieties on the surface or throughout the bulk of the hydrogel scaffold can significantly improve cellular attachment. For instance, in one embodiment, an RGD (arginine-glycine-aspartic acid) adhesion peptide sequence can be incorporated into the hydrogel described herein to facilitate cellular attachment. Inclusion of RGD domains in hydrogels can improve cellular migration, proliferation, growth, and organization in tissue regeneration applications (see, e.g., Shin, H. and Mikos, A. G., Biomaterials, 24: 4353-4364 (2003) and Hersel et al., Biomaterials, 24: 4385-4415 (2003)). In addition, a variety of cells have been shown to favorably bind to RGD-modified hydrogel scaffolds, including, for example, endothelial cells (ECs), fibroblasts, smooth muscle cells (SMCs), chondrocytes, and osteoblasts (see, e.g., Langer, R. and Tirrell, D. A., Nature, 425: 487-492 (2004); and El-Serbiny and Yacoub, supra)

For tissue engineering, a hydrogel may be selected to meet a number of design criteria to effectively mimic the extracellular matrix (ECM) and thereby promote new tissue formation. Such design criteria may include, but are not limited to, the ability to provide a 3D architecture for cell growth, biodegradability, porosity, proper surface chemistry, biocompatibility, cell adhesion, and enhanced vascularization (see, e.g., El-Serbiny and Yacoub, supra). “Extracellular matrix (ECM)” is well known in the art as the non-cellular component present within all tissues and organs that provides structural support to cells and performs other important functions. ECM is composed of an interlocking meshwork of fibrous proteins, including collagen, elastin, fibronectin, and laminin as well as polysaccharides such as glycosaminoglycans (GAGs), which typically form proteoglycans upon covalent linkage to proteins (see, e.g., Alberts et al, Molecular Biology of the Cell, Garland Science, London (2007)).

As described herein, the hydrogel may be generated using any material suitable for tissue engineering applications. For example, the hydrogel described herein may be generated using natural polymers, such as polynucleotides, polypeptides, and polysaccharides. Such natural polymers may be obtained or derived from any natural source, including, for example, a living organism (a mammal, a fish, an insect, or a plant). For example, chitosan is a natural polymer obtained from shellfish exoskeletons, while collagen is a natural polymer obtained from mammals. Other natural polymers that may be used in hydrogels include, but are not limited to, hyaluronic acid (HA), an amphiphilic peptide, alginate, collagen, fibrin, gelatin, chondroitin sulfate, carboxymethylcellulose, dextran, agarose carbomer, and derivatives thereof. It will be appreciated that hydrogels based on natural polymers are particularly suited for tissue engineering applications due to their intrinsic characteristics of biological recognition (e.g., presentation of receptor-binding ligands and susceptibility to cell-triggered proteolytic remodeling and degradation).

In some embodiments, the hydrogel of the present disclosure may be generated using a synthetic polymer. Examples of suitable synthetic polymers include, but are not limited to, poly(ethylene glycol) (PEG), poly(ethylene glycol) diacrylate (PEGDA), poly(lactic acid) (PLA), poly(ethylene oxide) (PEO), polyvinyl alcohol) (PVA), poly(hydroxyl-ethyl methacrylate) (PHEMA), methacrylated dextran-graft-lysine (Dex-MA-LA), methacrylamide-modified gelatin (Gel-MA), and derivatives thereof. Hydrogels based on synthetic polymers may exhibit less immunogenicity then natural polymer-based hydrogels, and may provide greater control over material characteristics and tissue responses.

In one embodiment, the hydrogel comprises hyaluronic acid (HA). HA is a linear polysaccharide and is the only non-sulfated glycosaminoglycan found distributed throughout the ECM, including lymphoid tissues (Jackson, D. G., Immunol. Rev., 230: 216-231 (2009)). Hyaluronic acid impacts cell motility and adhesion, differentiation, gene expression, and proliferation (Toole, B. P., Nat. Rev. Cancer 4, 528 (2004); Entwistle et al., J. Cell. Biochem., 61, 569-577 (1996); and Ponta et al., Nat. Rev. Mol. Cell Biol., 4: 33 (2003)). HA also can be easily modified through tunable chemistry, enabling the addition of adhesive ligands (Lei et al., Biomaterials, 32: 39-47 (2011); Shu et al., J Biomed. Mater. Res., Part A, 79: 902-912 (2006)) conjugation of drugs or growth factors (Peattie et al., Biomaterials, 25: 2789-2798 (2004)), and control of the elastic modulus and porosity of the hydrogel containing HA (Li et al., FASEB J., 27: 1127-1136 (2013); and Tan et al., Biomaterials, 30, 6844-6853 (2009)). HA and its derivatives have been clinically used as medical products for over three decades (Kuo, J. W., Practical Aspects of Hyaluronan Based Medical Products, Boca Raton: CRC/Taylor & Francis; (2006)). More recently, HA has become recognized as an important building block for the creation of new biomaterials with utility in tissue engineering and regenerative medicine. HA-containing hydrogels and methods of producing HA-containing hydrogels are described in, e.g., Burdick, J. A. and G. D. Prestwich, Adv Mater., 23(12): H41-H56 (2011); and Xu et al., Soft Matter, 8(12): 3280-3294 (2012). In some embodiments, the hydrogel comprises hyaluronic acid crosslinked with polyethylene glycol diacrylate (PEGDA). Any suitable amount of PEGDA may be incorporated into the HA hydrogel. For example, the volume ratio of hyaluronic acid to PEGDA in the hydrogel may be about 4:1.

HA hydrogels described herein can be prepared using any suitable method known in the art. Such methods may include, for example, emulsification, lyophilization, emulsification-lyophilization, spray drying, solvent casting-leaching, gas foaming-leaching, photolithography, photocrosslinking, electrospinning, microfluidics, micromolding, and 3D-organ/tissue printing (see, e.g., El-Serbiny and Yacoub, supra, and Xu et al., supra).

The hydrogel may be conjugated with one or more molecules or substances that provide stimulatory signals required for T cell activation. In this regard, the hydrogel composition may be conjugated with a first molecule that provides a first T cell activating signal and a second molecule that provides a second T cell activating signal. The terms “T cell activation” and “T cell stimulation” are used interchangeably herein and refer to an antigen-dependent process leading to proliferation and differentiation of naive T cells into effector cells. This process requires primary and coactivating signals triggering intracellular signal transduction cascades and new gene expression. In particular, activation of naive precursor T cells requires the generation of two signals. “Signal 1” occurs when the T cell receptor (TCR) binds a foreign antigenic protein on the cell surface of an antigen-presenting cell (APC) or a target cell, and the T cell coreceptor binds the major histocompatibility complex (MHC) molecule on the APC or target cell. “Signal 2” occurs when co-activating molecules on the T cell bind co-stimulatory proteins on the APC or target cell, the most important of which is the B7 protein. B7 binding by T cell co-stimulatory proteins results in T cell activation, whereas a lack of binding results in apoptosis of the cell. The combination of signals 1 and 2 determines the nature of the T cell's response to the antigen. Activation of cytotoxic (CD8) T cells via MHC Type I binding results in the direct lysis of target cells, whereas activation of helper (CD4) T cells via MHC Type II binding causes multiple downstream effects including synthesis of important pro-inflammatory molecules (cytokines) such as tumor-necrosis factor; enhancement of antibody secretion by B cells, and enhanced killing by cytotoxic CD8 cells. The combined actions of signal 1 and signal 2 stimulate the T cell to proliferate and begin to differentiate into an effector cell (Bretscher, P., Proc Natl Acad Sci USA, 96(1): 185-190 (1999); Alberts et al., Molecular Biology of the Cell, 4^{th} Ed., New York: Garland Science (2002)).

In certain embodiments, the first molecule that provides a first T cell activating signal (i.e., “signal 1”) comprises an antigen presenting complex. “Antigen presenting complexes” comprise an antigen binding cleft, which harbors an antigen for presentation to a T cell or T cell precursor. Antigen presenting complexes can be, for example, MHC class I or class II molecules, and can be linked or tethered to provide dimeric or multimeric MHC. Thus, in some embodiments, the antigen presenting complex comprises a peptide antigen in the context of MHC class I or II molecular complex. The MHC molecular complex may be monomeric or dimeric. Dimeric MHC class I constructs can be constructed by fusion to immunoglobulin heavy chain sequences, which are then associated through one or more disulfide bonds (optionally with associated light chains).

In some embodiments, MHC class I molecular complexes may comprise at least two fusion proteins. A first fusion protein comprises a first MHC class I a chain and a first immunoglobulin heavy chain (or portion thereof comprising the hinge region), and a second fusion protein comprises a second MHC class I a chain and a second immunoglobulin heavy chain (or portion thereof comprising the hinge region). The first and second immunoglobulin heavy chains associate to form the MHC class I molecular complex, which comprises two MHC class I peptide-binding clefts. The immunoglobulin heavy chain can be the heavy chain of an IgM, IgD, IgGl, IgG3, IgG2, IgG2a, IgG4, IgE, or IgA. In some embodiments, an IgG heavy chain is used to form MHC class I molecular complexes. If multivalent MHC class I molecular complexes are desired, IgM or IgA heavy chains can be used to provide pentavalent or tetravalent molecules, respectively.

Exemplary MHC class I molecules include HLA-A, HLA-B, HLA-C, and HLA-E, and these may be employed individually or in any combination. In some embodiments, the antigen presenting complex comprises an HLA-A2 ligand.

MHC class II molecular complexes may comprise at least four fusion proteins. Two first fusion proteins comprise (i) an immunoglobulin heavy chain (or portion thereof comprising the hinge region) and (ii) an extracellular domain of an MHC class IIβ chain. Two second fusion proteins comprise (i) an immunoglobulin κ or λ light chain (or portion thereof) and (ii) an extracellular domain of an MHC class IIα chain. The two first and the two second fusion proteins associate to form the MHC class II molecular complex. The extracellular domain of the MHC class IIβ chain of each first fusion protein and the extracellular domain of the MHC class IIα chain of each second fusion protein form an MHC class II peptide binding cleft. Exemplary MHC class II molecular complexes are described in U.S. Pat. Nos. 6,458,354, 6,015,884, 6,140,113, and 6,448,071.

Fusion proteins of an MHC class II molecular complex can comprise a peptide linker inserted between an immunoglobulin chain and an extracellular domain of an MHC class II polypeptide. The length of the linker sequence can vary, depending upon the flexibility required to regulate the degree of antigen binding and receptor cross linking.

The antigen presenting complex may present a peptide antigen for activation of T cells (CD8+ or CD4+ T cells). A variety of peptide antigens can be bound to antigen presenting complexes. The nature of the antigen depends on the type of antigen presenting complex that is used. For example, peptide antigens can be bound to MHC class I and class II peptide binding clefts. Non-classical MHC-like molecules can be used to present non-peptide antigens such as phospholipids, complex carbohydrates, and the like (e.g., bacterial membrane components such as mycolic acid and lipoarabinomannan). Any peptide capable of inducing an immune response can be bound to an antigen presenting complex. Antigenic peptides include cancer-specific or cancer-associated antigens, cancer neoantigens, autoantigens, alloantigens (e.g., blood group antigens and histocompatibility antigens), and antigens of infectious agents.

The terms “cancer-specific antigen (CSA)” and “tumor-specific antigen (TSA)” are used interchangeably herein and refer to a protein, carbohydrate, or other molecule that is uniquely expressed by and/or displayed on cancer cells and is not expressed by or displayed on other cells in the body (e.g., normal healthy cells). In contrast, the terms “cancer-associated-antigen (CAA)” and “tumor-associated-antigen (TAA)” are used interchangeably herein and refer to a protein, carbohydrate, or other molecule that is not uniquely expressed by or displayed on a tumor cell and instead also is expressed on normal cells under certain conditions. Cancer-specific antigens and cancer-associated antigens are well known in the art. In some embodiments, the CSA or CAA comprises one or more antigenic cancer epitopes associated with a malignant cancer or tumor, a metastatic cancer or tumor, or a leukemia. A cancer “neoantigen” is a novel cancer-specific antigen that arises as a consequence of tumor-specific mutations (T. N. Schumacher and R. D. Schreiber, Science, 348(6230):69-74 (2015); and T. C. Wirth and F. Kühnel, Front Immunol., 8: 1848 (2017)).

“Antigens of infectious agents” include components of protozoa, bacteria, fungi (both unicellular and multicellular), viruses, prions, intracellular parasites, helminths, and other infectious agents that can induce an immune response. Bacterial antigens include antigens of gram-positive cocci, gram positive bacilli, gram-negative bacteria, anaerobic bacteria, such as organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma. Antigens of protozoan infectious agents include antigens of malarial Plasmodia species, Leishmania species, Trypanosoma species and Schistosoma species. Fungal antigens include antigens of Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia. Viral peptide antigens include, but are not limited to, those of adenovirus, herpes simplex virus, papilloma virus, respiratory syncytial virus, poxviruses, HIV, influenza viruses, and CMV. Particularly useful viral peptide antigens include HIV proteins such as HIV gag proteins (including, but not limited to, membrane anchoring (MA) protein, core capsid (CA) protein and nucleocapsid (NC) protein), HIV polymerase, influenza virus matrix (M) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like.

Antibodies directed against self- or autoantigens (“autoantibodies”) have been directly associated with the pathophysiology of certain autoimmune diseases. Autoantigens are described in, e.g., Backes et al., BMC Genomics, 12: 340 (2011); Atassi, M. Z. and P. Casali, Autoimmunity, 41(2): 123-32 (2008); and Wu et al., Ann N Y Acad Sci., 1050:134-45 (2005).

It is known in the art that the antigen receptor molecules on human T lymphocytes are noncovalently associated on the cell surface with the CD3 (T3) molecular complex. Perturbation of this complex with anti-CD3 monoclonal antibodies induces T cell activation. It will be appreciated, therefore, that resting T cells can be activated by incubation with an anti-CD3 antibody (i.e., “signal 1” as described above). Thus, in some embodiments, the first molecule may be an anti-CD3 antibody.

The hydrogel composition also may be conjugated with a second molecule that provides a second T cell activating signal (“signal 2”). The second molecule may be any suitable molecule or compound capable of activating resting or naïve T cells. In some embodiments, the second molecule is a co-stimulatory molecule. The term “co-stimulatory molecule,” as used herein, refers to a molecule that has a biological effect on a precursor T cell, a naive T cell, or on an antigen-specific T cell. Such molecules include, but are not limited to, molecules that specifically bind to CD28 (including antibodies), CD80 (B7-1), CD86 (B7-2), B7-H3, 4-1BB, 4-1BBL, CD27, CD30, CD 134 (OX-40L), B7h (B7RP-1), CD40, inducible co-stimulator (ICOS), and LIGHT, antibodies that specifically bind to HVEM, antibodies that specifically bind to CD40L, antibodies that specifically bind to OX40, and antibodies that specifically bind to 4-1BB. T cell co-stimulatory molecules are further described in, e.g., Chen, L. & D. B. Flies, Nature Reviews Immunology, 13:227-242 (2013). In one embodiment, the second molecule is an antibody that specifically binds to CD28. CD28 costimulation plays an important role during T cell activation to promote T cell survival, augment production of cytokines, and overcome T cell anergy (Alegre et al., Nat Rev Immunol., 1: 220-228 (2001); June et al., Mol Cell Biol., 7: 4472-4481 (1987)).

In certain embodiments, the hydrogel may be conjugated with one or more anti-CD3 antibodies and one or more anti-CD28 antibodies. In vitro T cell stimulation using anti-CD3 and/or anti-CD28 antibodies is described in, e.g., Trickett, A. and Kwan, Y. L., J. Immunol Methods, 275(1-2):251-5 (2003); Tsoukas et al., J Immunol., 135(3): 1719-172 (1985); Hombach et al., Cancer Res., 61(5): 1976-1982 (2001); and Nijhuis et al., Cancer Immunol Immunother., 32: 245-250 (1990)).

The one or more anti-CD3 antibodies and one or more anti-CD28 antibodies may be any suitable type of antibody, or antigen-binding fragment thereof. In one embodiment, each of the one or more anti-CD3 antibodies and one or more anti-CD28 antibodies is a monoclonal antibody. The term “monoclonal antibodies,” as used herein, refers to antibodies that are produced by a single clone of B-cells and bind to the same epitope. In another embodiment, each of the one or more anti-CD3 antibodies and one or more anti-CD28 antibodies is a polyclonal antibody. The term “polyclonal antibodies” refers to a population of antibodies that are produced by different B-cells and bind to different epitopes of the same antigen. The terms “fragment of an antibody,” “antibody fragment,” “functional fragment of an antibody,” and “antigen-binding portion” are used interchangeably herein to mean one or more fragments or portions of an antibody that retain the ability to specifically bind to an antigen (see, generally, Holliger et al., Nat. Biotech., 23(9): 1126-1129 (2005)). Examples of antibody fragments include, but are not limited to, (i) a Fab fragment, which is a monovalent fragment consisting of the variable domains of the antibody heavy (VH) and light chains (VL) and the constant domains of the antibody heavy (CH) and light chains (CL); (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are described in more detail in, e.g., U.S. Patent Application Publication 2009/0093024 A1.

In some embodiments, antibodies may be conjugated to the HA hydrogel via a linker molecule. For example, the one or more anti-CD3 and/or anti-CD28 antibodies may be linked to the HA hydrogel with a linker molecule comprising a disulfide bond. The linker molecule may be cleavable and may comprise a reactive chemical group that can react with the HA hydrogel and a reactive chemical group that can react with the one or more antibodies, such as, for example, N-succinimidyl esters and N-sulfosuccinimidyl esters. In other embodiments, a non-cleavable linker may be used. A non-cleavable linker may comprise any chemical moiety that is capable of linking the one or more antibodies to an HA hydrogel in a stable, covalent manner. Thus, non-cleavable linkers may be substantially resistant to acid-induced cleavage, light-induced cleavage, peptidase-induced cleavage, esterase-induced cleavage, and disulfide bond cleavage, at conditions under which the one or more antibodies remain active. Many suitable crosslinking reagents that form non-cleavable linkers between biomolecules are well known in the art. Biomolecules, such as antibodies, may be conjugated to the HA hydrogel using other appropriate coupling reactions, including, for example, succinimide with amine, carboxylic acid with amine, Michael's addition, and maleimide with thiol. Other conjugation chemistries that may be used in connection with the present disclosure include iodinate with thiol, click reaction of azide with alkyne, and oxime ligation of aldehyde with amine (see, e.g., Jabarri, E., Curr. Opin. Biotechnol., 22(5): 655-660 (2011); Shendi et al., J. Mater. Chem. B, 4: 2803-2818 (2016); and Camci-Unal et al., Soft Matter, 6(20): 5120-5126 (2010)). In one embodiment, the one or more antibodies may be conjugated to hyaluronic acid in the hydrogel using thiol-diene chemistry. In such embodiments, therefor, the hyaluronic acid, one or more anti-CD3 antibodies, and one or more anti-CD28 antibodies are thiolated. Thiol modification of biomolecules, such as HA and antibodies, may be performed using routine methods known in the art.

The density of T cell stimulating signals is an important parameter to control and optimize on biocompatible tissue matrices. It has been shown that effective T cell stimulation is observed when the inter-ligand spacing is maintained below 75-150 nm on particle and planar surfaces (Hickey et al., Nano Lett., 17 (2017); Deeg et al., Nano Lett., 13: 5619-5626 (2013); and Matic et al., Nano Lett., 13: 5090-5097 (2013)). Thus, the one or more anti-CD3 and anti-CD28 antibodies may be included in the hydrogel composition in any suitable concentration that provides for optimal T cell activation. In some embodiments, for example, the concentration of each of the one or more anti-CD3 antibodies and/or the one or more anti-CD28 antibodies ranges from about 0.1 μg/mL to about 20 μg/mL, including 0.1, 0.2, 0.3, 0.4, 0.5, 1, 2, 3, 4, 5, 6, 7, 8, 9, 10, 11, 12, 13, 14, 15, 16, 17, 18, 19, and 20 μg/mL, in some embodiments, less than about 4 μg/mL, in some embodiments, about 1 μg/mL to about 4 μg/mL (e.g., 1.5 μg/mL, 2.0 μg/mL, 2.5 μg/mL, 3.0 μg/mL, or 3.5 μg/mL). Including each of the anti-CD3 and CD28-antibodies in such amounts allows for spacing between antibodies on the aTM surface of at least 500 nm.

The addition of cell-adhesive ligands has been shown to increase cell attachment to various surfaces (Massia, S. P. & Hubbell, J. A., J. Cell Biol., 114: 1089-1100 (1991)). As such, the disclosed hydrogel composition may comprise additional ECM-binding proteins. Any suitable ECM-binding protein may be incorporated into the HA hydrogel, such as, for example, collagens, elastins, fibronectins, and laminins (Alberts et al., supra). For instance, in one embodiment, an RGD (arginine-glycine-aspartic acid) adhesion peptide sequence can be incorporated into the hydrogel described herein to facilitate cellular attachment. More generally, in some embodiments, the hydrogel comprises a cell adhesion peptide. In particular embodiments, the cell adhesion peptide is selected from one or more from the group including a RGD peptide, cyclic RGD peptide, YIGSR peptide, and IKVAV peptide.

As discussed above, inclusion of RGD domains in hydrogels can improve cellular migration, proliferation, growth, and organization in tissue regeneration applications (see, e.g., Shin and Mikos, supra, and Hersel et al., supra)). In one embodiment, the hydrogel comprises a cyclic RGD peptide (e.g., CCRRGDWLC (SEQ ID NO: 1)). In some embodiments, the hydrogel comprises a cell adhesion protein selected from a group including laminin, collagen, fibronectin, fibrinogen, and fibrin. In particular embodiments, the hydrogel comprises a laminin protein. Laminins are biologically active extracellular matrix (ECM) proteins composed of heterotrimers formed by one heavy chain (a) and two light chains (β and γ) that combine to form fourteen unique isoforms (see, e.g., Aumailley M., Cell Adhesion & Migration, 7(1): 48-55 (2013)). Laminins can self-assemble, bind to other matrix macromolecules, and have unique and shared cell interactions mediated by integrins, dystroglycan, and other receptors. Through these interactions, laminins contribute to cell differentiation, cell shape and movement, maintenance of tissue phenotypes, and promotion of tissue survival (see, e.g., Colognato, H. and Yurchenco, P. D., Dev. Dyn., 218: 213-234 (2000); and Beck et al., The FASEB Journal, 4(2): 148-160 (1990)).

Lymph nodes are known to contain long collagen-based reticular fiber networks. To mimic this unique structure, the disclosed HA hydrogel may further comprise polymeric nanofibers. The polymeric nanofibers can be comprised of any of a variety of synthetic and/or natural polymers. Examples of natural polymers include collagen, cellulose, silk fibroin, keratin, gelatin and polysaccharides such as chitosan and alginate (Vasita, R. and Katti, D., Int. J. Nanomed., 1(1): 15-30 (2006); and Khajavi et al., J. Appl. Polym. Sci., 133(3) (2016)). Examples of synthetic polymers include poly(lactic acid) (PLA), polycaprolactone (PCL), including poly(ε-caprolactone), polyurethane (PU), polyglycolide, poly(lactic-co-glycolic acid) (PLGA), poly(3-hydroxybutyrate-co-3-hydroxyvalerate) (PHBV), and poly(ethylene-co-vinylacetate) (PEVA) (Vasita, supra; and Khajavi et al., supra). In one embodiment, the hydrogel comprises poly-caprolactone (PCL) nanofibers. PCL is a synthetic biodegradable aliphatic polyester which has attracted considerable attention in recent years, notably in the biomedical areas of controlled-release drug delivery systems, absorbable surgical sutures, nerve guides, and three-dimensional (3-D) scaffolds, for use in tissue engineering. Various polymeric devices like microspheres, microcapsules, nanoparticles, pellets, implants, and films have been fabricated using PCL. PCL can be transformed by spinning into filaments for subsequent fabrication of desirable textile structures (Azimi et al., Journal of Engineered Fibers and Fabrics, 74: 9(Issue 3): 74-90 (2014)).

Polymeric nanofibers can be produced by a number of different techniques utilizing physical, chemical, thermal, and electrostatic fabrication techniques such as bacterial cellulose, super drawing, templating, phase separation, vapor-phase polymerization, self-assembly, kinetically controlled solution synthesis, electrospinning, novel modular meltblowing and centrifugal spinning.

The above-described hydrogel composition may be used in a variety of in vitro and in vivo applications to activate and stimulate T cells. In some embodiments, the disclosure provides a method of activating T cells which comprises (a) contacting one or more T cells with the above-described composition comprising a hydrogel; and (b) culturing the one or more T cells and the composition under conditions whereby the one or more T cells proliferate and differentiate, for example, into effector T cells. The T cell may be any T cell, such as a cultured T cell, e.g., a primary T cell, or a T cell from a cultured T cell line, or a T cell obtained from a mammal. In one embodiment, the T cells that are contacted with the hydrogel composition are naïve T cells. The term “naïve T cell,” as used herein, refers to a T cell that has not yet encountered and responded to a specific antigen, and includes T cells leaving the thymus. If obtained from a mammal, the T cell can be obtained from numerous sources, including but not limited to blood, bone marrow, lymph node, the thymus, or other tissues or fluids. T cells can also be enriched for or purified. In one embodiment, The T cell preferably is a human T cell (e.g., isolated from a human). T cell lines are available from, e.g., the American Type Culture Collection (ATCC, Manassas, Va.), and the German Collection of Microorganisms and Cell Cultures (DSMZ) and include, for example, Jurkat cells (ATCC TIB-152), Sup-Tl cells (ATCC CRL-1942), RPMI 8402 cells (DSMZ ACC-290), Karpas 45 cells (DSMZ ACC-545), and derivatives thereof.

“Effector” lymphocytes, such as effector T cells, mediate the removal of pathogens from an organism (e.g., a human), without the need for further differentiation, as opposed to naïve lymphocytes, which must differentiate and/or proliferate before becoming effector cells. With respect to T cells, all effector functions involve the interaction of an armed effector T cell with a target cell displaying specific antigen. The term “armed effector T cell,” as used herein, refers to a T cell that can be triggered to perform an effector function immediately on contact with cells bearing the peptide:MHC complex for which they are specific. The effector proteins released by these T cells are focused on the appropriate target cell by mechanisms that are activated by recognition of antigen on the target cell. Different types of effector T cells are specialized to deal with different types of pathogen, and the effector molecules they are programmed to produce cause distinct and appropriate effects on the target cell. The three types of effector T cells include (1) CD8 cytotoxic cells (also referred to as “CD8+ T cells,” “cytotoxic T cells,” and “cytotoxic T lymphocytes (CTLs”)), which kill target cells that display peptide fragments of cytosolic pathogens, most notably viruses, bound to MHC class I molecules at the cell surface, (2) TH1 cells, and (3) TH2 cells (also referred to as “T helper cells,” “CD4 T cells,” and “CD4+ T cells”). Both TH1 and TH2 cells express the CD4 co-receptor and recognize fragments of antigens degraded within intracellular vesicles, displayed at the cell surface by MHC class II molecules. TH1 cells activate macrophages, enabling them to destroy intracellular microorganisms more efficiently; they can also activate B cells to produce strongly opsonizing antibodies belonging to certain IgG subclasses (IgG1 and IgG3 in humans, and their homologues IgG2a and IgG2b in the mouse). TH2 cells, on the other hand, drive B cells to differentiate and produce immunoglobulins of all other types, and are responsible for initiating B-cell responses by activating naive B cells to proliferate and secrete IgM. Effector T cell properties and functions are described in detail in, e.g., C. A. Janeway et al. (eds.), Immunobiology, 9th Ed., Garland Publishing, New York, N.Y. (2016). In some embodiments, the effector T cells are CD8+ T cells and/or CD4+ T cells, such as, for example, a CD4+/CD8+ double positive T cell, a CD4+ helper T cell, e.g., Th, and Th2 cells, a CD8+ T cell (e.g., a cytotoxic T cell), and the like. In one embodiment, the effector T cell is a CD8+ T cell or a CD4+ T cell.

In some embodiments, the effector cells may be other than T cells. Effector cells also include, but are not limited to, effector B lymphocytes, natural killer (NK) cells, dendritic cells, and mesenchymal stromal cells. Furthermore, the methods described herein may also be used to stimulate the proliferation and differentiation of chimeric antigen receptor-expressing T cells (CAR-T cells).

In other embodiments, the compositions and methods described herein may be used to generate memory lymphocytes. The terms “memory lymphocytes” and “memory cells” are used interchangeably and refer to lymphocytes that mediated immunological memory. Memory lymphocytes are more sensitive to antigen than are naïve lymphocytes and respond rapidly on reexposure to the inducing antigen. Memory B cells are generated in germinal center (GC) reactions in the course of T cell-dependent immune responses and are distinguished from naïve B cells by an increased lifespan, faster and stronger response to stimulation, and expression of somatically mutated and affinity matured immunoglobulin (Ig) genes (Seifert, M. and R. Kuppers, Leukemia, 30(12): 2283-229 (2016)). Memory T cells are a subset of T cells that have previously encountered and responded to a cognate antigen. Memory T cells can recognize foreign invaders, such as bacteria or viruses, as well as cancer cells, and may be either CD4+ or CD8+ and usually express CD45RO (Akbar et al., Journal of Immunology, 140 (7): 2171-8 (1988)).

In certain embodiments, the methods described herein comprise culturing the one or more T cells and the hydrogel composition under conditions whereby the one or more T cells proliferate and differentiate, for example, into effector T cells. Methods for selecting suitable T lymphocytes and methods for culture, amplification, screening, and purification of such cells are known in the art.

The one or more T cells may be contacted with the hydrogel composition ex vivo, in vivo, or in vitro. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. The methods described herein desirably involve ex vivo and in vivo components.

In this regard, for example, the isolated T cells described above can be cultured with the hydrogel composition ex vivo or in vitro under conditions such that the one or more T cells proliferate and differentiate, for example, into effector T cells, and then directly transferred into a diseased mammal (preferably a human) in need thereof. Such a cell transfer method is referred to in the art as “adoptive cell transfer (ACT),” in which immune-derived cells are passively transferred into a new recipient host to transfer the functionality of the donor immune-derived cells to the new host. Adoptive cell transfer therapy typically is used in the art to treat different types of cancer and autoimmune diseases. Thus, the disclosure also provides methods of killing cancer cells in a subject (e.g., a human). In one embodiment, the method comprises: (a) contacting one or more T cells isolated from a subject with the hydrogel composition described herein; (b) culturing the one or more T cells under conditions whereby the one or more T cells proliferate and differentiate, e.g., are activated, for example, into effector T cells; and (c) administering the activated T cells to the subject, whereby a cancer-specific immune response is induced in the subject and the cancer cells are killed. Descriptions of the hydrogel composition, effector T cells, T cell culture conditions, and components thereof, set forth above also are applicable to those same aspects of the aforementioned method of killing cancer cells. In certain embodiments, the one or more T cells are cultured in the presence of a cancer-specific antigen, as described herein.

Adoptive cell transfer methods to treat various types of cancers are known in the art and are disclosed in, for example, Gattinoni et al., Nat. Rev. Immunol, 6(5): 383-393 (2006); June, C H, J. Clin. Invest., 117(6): 1466-76 (2007); Rapoport et al., Blood, 117(3): 788-797 (201 1); and Barber et al., Gene Therapy, 18: 509-516 (2011)). The effector T cells administered to the subject (e.g., a human) can be allogeneic or autologous to the subject. In “autologous” administration methods, cells (e.g., lymphocytes) are removed from a mammal, stored, engineered or modified (as described herein) and returned back to the same mammal. In “allogeneic” administration methods, a mammal receives cells (e.g., blood-forming stem cells or lymphocytes) from a genetically similar, but not identical, donor. Ideally, the cells are autologous to a human subject.

In other embodiments, the hydrogel composition described herein may be administered directly to a subject (e.g., a human), such as a subject suffering from cancer. In this regard, the disclosure provides a method of killing cancer cells in a subject (i.e., in vivo), which method comprises administering the disclosed hydrogel composition to the subject, whereby one or more T cells in the subject proliferate and differentiate into effector T cells and/or memory T cells specific for the cancer and the cancer cells are killed. Descriptions of the hydrogel composition, effector T cells, memory T cells, and components thereof, set forth above also are applicable to those same aspects of the aforementioned method of killing cancer cells in a subject. In such embodiments, circulating T cells from a subject's body can percolate through the hydrogel and become activated or stimulated. Such methods can be used to treat a condition or disorder that is antigen-specific (as in cancer or infectious diseases) or more generally immunostimulatory fashion (HIV or other immune targeting viruses). In such embodiments, the hydrogels can be administered subcutaneously, intraperitoneally, or intratumorally with or without added T cells.

The methods described herein may be performed to kill cancer cells of any type, particularly epithelial cell cancers (i.e., carcinomas). Examples of such cancers include, but are not limited to, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (e.g., a glioma, glioblastoma (also known as glioblastoma multiforme) astrocytoma, CNS lymphoma, germinoma, medulloblastoma, Schwannoma craniopharyogioma, ependymoma, pineaioma, hemangioblastoma, acoustic neuroma, oligodendroglioma, menangioma, neuroblastoma, retinoblastoma, and brain metastases). In certain embodiments, the cancer is melanoma, leukemia, multiple myeloma, or prostate cancer.

In other embodiments, the cancer may be a hematological cancer. Examples of hematological cancers that may be treated by the methods disclosed herein include, but are not limited to, leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukemia, and myelodysplasia.

Activated effector T cells or the hydrogel, as described herein, can be formulated into a composition, such as a pharmaceutical composition, and administered to a human. For example, the pharmaceutical composition can comprise a population of effector T cells or the hydrogel composition. When T cells or the hydrogel are administered in a composition or formulation, the T cells or the hydrogel are either administered to a site of treatment or may localize at a site of treatment (e.g., cell type, tissue type, etc.).

The pharmaceutical composition desirably comprises a carrier, such as a pharmaceutically acceptable carrier. For example, the pharmaceutical composition may contain preservatives, such as, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition. In addition, buffering agents may be used in the composition. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition. Methods for preparing administrable (e.g., parenterally administrable) compositions are known to those skilled in the art and are described in more detail in, for example, Remington: The Science and Practice of Pharmacy, Lippincott Williams & Wilkins; 21st ed. (May 1, 2005).

Ideally, the killing of cancer cells by the methods described herein results in the treatment of the cancer. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the methods described herein comprise administering a “therapeutically effective amount” of the hydrogel composition or a composition comprising effector T cells. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the hydrogel or effector T cells to elicit a desired response in the individual. For example, a therapeutically effective amount of the hydrogel or effector T cells is an amount which induces a sufficient cancer-specific immune response to destroy cancer cells.

Alternatively, the pharmacologic and/or physiologic effect may be prophylactic, i.e., the effect completely or partially prevents a disease or symptom thereof. In this respect, the method comprises administering a “prophylactically effective amount” of the composition comprising the hydrogel or effector T cells to a mammal (e.g., a human) that is predisposed to cancer. A “prophylactically effective amount” refers to an amount effective, at dosages and for periods of time necessary, to achieve a desired prophylactic result (e.g., prevention of disease onset).

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the disclosure. The desired dosage can be delivered by a single bolus administration of the composition, by multiple bolus administrations of the composition, or by continuous infusion administration of the composition.

The compositions described herein can be administered to a subject using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

In some embodiments, the compositions described herein may be co-administered with T cells isolated from the subject receiving the composition (autologous), or T cells isolated from a donor other than the subject (allogeneic). In other embodiments, the compositions described herein may be co-administered with other anti-cancer agents to improve the therapeutic efficacy of treatment. The clinical regimen for co-administration may encompass co-administration at the same time, before or after the administration of the other component. Particular combination therapies include chemotherapy, radiation, surgery, hormone therapy, or other types of immunotherapy. Chemotherapuetic agents that may be used in combination with the disclosed methods include, for example, mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Specific chemotherapeutic agents include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, fuldarabine, navelbine, farnesyl-protein transferase inhibitors, transplatinum, 5-fluorouracil, vincristin, and vinblastin, or any analog or derivative of the foregoing or combinations thereof.

In some embodiments, the compositions described herein may be co-administered with radiotherapy, methods of which are understood in the field. In some embodiments, radiotherapy is employed before, during and/or after administration of the compositions described herein.

In some embodiments, the compositions described herein may be administered before, during, and/or after surgery. Surgeries include resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that embodiments herein may be used in conjunction with removal of superficial cancers, precancers, or incidental amounts of normal tissue.

In some embodiments, the compositions described herein are provided as part of a kit or system along with one or more additional components, such as instructions, devices for administration, additional therapeutic agents, diagnostic agents, research agents, etc.

The following examples further illustrate the invention but, of course, should not be construed as in any way limiting its scope.

### EXAMPLES

The following materials and methods were employed in the Examples described below.

### Mice

B6, 2C, and PMEL transgenic mice were maintained per guidelines approved by the Johns Hopkins University's Institutional Review Board.

### Reagents

Soluble MHC-Ig dimers loaded with peptides (pMHC-Ig) and artificial antigen presenting cells (aAPC) were produced as described (Hickey et al., Nano Lett., 17 (2017); and Kosmides et al., supra).

### Hydrogel Preparation

Thiol-modified hyaluronic acid (HA) (ESI BIO, Alameda, Calif., USA) was resuspended with 1 mL sterile dH₂O and incubated at 37° C. for 30 minutes until completely dissolved to form 1% HA solution in 1×PBS. To form hydrogels, HA was plated immediately after mixing with polyethylene glycol diacrylate (PEGDA) with a molecular weight of 3400 (Laysan Bio, Arab, Ala.) crosslinker at a 4:1 volume ratio to fully cover the well. Plated hydrogels were incubated for a minimum of 1 hour prior to cell culture.

### Preparation of aTM

HA solution was prepared as previously described. Anti-CD3 and anti-CD28 antibodies were purchased respectively from BioXCell (145-2C11; West Lebanon, N.H., USA) and BioLegend (37.51; San Diego, Calif., USA). Antibody and MHC-Ig dimers were partially reduced with 100 mM dithiothreitol (DTT) for 30 minutes at room temperature to expose free thiol groups and thoroughly washed through a centrifugation filtration with a 50-kDa MWCO filter. PEGDA crosslinker was added to reduced MHC-Ig dimers, anti-CD3 and anti-CD28 antibody solutions to a final concentration of 0.5% PEGDA for preparation for crosslinking. Prior to hydrogel formation, MHC-Ig dimers or anti-CD3 antibodies and anti-CD28 co-stimulatory signals with 0.5% PEGDA were added to the HA solution to directly attach signals on HA through thiol-diene chemistry. The HA-antibody solution was then mixed with PEGDA crosslinker at a 4:1 ratio to be plated. The aTM was allowed to form within flat-bottomed tissue culture plates to form a complete layer for at least 1 hour prior to washing 3 times with 1×PBS to remove any unbound stimulatory signal, and cells were subsequently plated. To investigate the effects of extracellular matrix (ECM) protein attachment, cyclic RGD (CCRRGDWLC (SEQ ID NO: 1)), which was synthesized by solid phase methods as described previously (Li et al., Stem Cells Transl. Med., 3: 662-670 (2014)), or laminin (ThermoFisher) were added to make a HA solution with protein concentration of 100 μM and 20 μg/mL, respectively, prior to the hydrogel formation. To investigate the stiffness effects of aTMs, the PEGDA crosslinker concentration was changed from a final concentration of 0.05 wt/vol % to 2 wt/vol %.

### Characterization of aTM

To evaluate the mechanical stiffness of aTMs, elastic moduli of hydrogels were measured using an Ares G2 oscillatory shear rheometer. First, HA solution was mixed with varying PEGDA crosslinker concentrations to a final volume of 200 μL and placed immediately on the stationary lower plate of the rheometer. The shear storage modulus, G′, and the sheer loss modulus, G″, were recorded during in situ hydrogel formation over one hour at 37° C. The elastic modulus, E′, was calculated by E′=2G′(1+γ) where γ is the Poisson's ratio. For HA hydrogels, γ was assumed to be 0.5 because the Poisson's ratio of incompressible materials is approximately 0.5 and the hydrogels are used under low strain conditions (Li et al., FASEB J., 27: 1127-1136 (2013). To estimate the density of the ligand on the surface of the aTM, the thickness observed by cells was assumed to be 1 μm, and then the density was calculated based off the total mass of Signal 1 and 2 within this slab and then dividing by the surface area.

### CD8+T Lymphocyte Isolation

Murine cells were obtained from adult mouse lymph nodes and spleens. Obtained cells were treated with ACK lysing buffer to lyse red blood cells and filtered through cell strainers to isolate splenocytes. PBMCs from healthy donors were isolated by Ficoll-Paque PLUS gradient centrifugation (GE Healthcare). CD8+T lymphocytes were then isolated from splenocytes or PBMCs by negative selection using CD8+ isolation kits and magnetic columns from Miltenyi Biotech (Auburn, Calif., USA) according to the manufacturer's protocol.

### Ex Vivo T Cell Culture and Activation

For ex vivo T cell expansion, isolated CD8+ T cells were cultured in the T cell culture media (RPMI supplemented with L-glutamine, non-essential amino acids, vitamin solution, sodium pyruvate, β-mercaptoethanol, 10% fetal bovine serum, ciproflaxin, and a cocktail of T cell growth factors as described previously (Oelke et al., Clin. Cancer Res., 6: 1997-2005 (2000)). In the case of human T cell expansion, 10% AB serum was used instead of 10% fetal bovine serum. On day 3 or 4 of culture, cells were fed with half the volume of the initial T cell culture media with twice the concentration of T cell growth factor cocktail. For activation with aAPC, T cells were co-cultured with a concentration of 75 pM-bound pMHC-Ig on the aAPC and then plated on respective surfaces. For stimulation on the aTM, cells were plated on aTM with concentrations of the stimulatory antibody (either anti-CD3 and anti-CD28 or pMHC-Ig and anti-CD28) conjugated to the HA hydrogel.

### T Cell Proliferation Assay

CD8+ T cells were isolated as previous described and resuspended in 1 mL T cell culture media. Cells were mixed with 1 μL CELLTRACE™ carboxyfluorescein succinimidyl ester (CFSE) dye (ThermoFisher) in 1 mL T cell culture media per 3 million cells and incubated at 37° C. for 20 minutes. CFSE stained cells were washed with 50 mL T cell culture media to remove unstained dye and plated. On day 3 of culture, cells were harvested and stained with a 1:100 PBS solution of APC-conjugated rat anti-mouse CD8a, clone 53-6.7 (BD Pharmingen) for 15 minutes at 4° C. The CFSE fluorescence intensity was measured using BD FACSCALIBUR™ flow cytometer. Cell proliferation was analyzed using FLOWJO™ with diluted CFSE fluorescence peaks signifying population after each round of cell division. A subset of the cells were allowed to expand for 7 days and viable cells were counted with a hemocytometer to determine fold expansion. Images of cell cultures were taken with an Olympus IX71 inverted light microscope at a 4× magnification on day 3 of cultures.

### Time Course Experiments

Two million purified CD8+ T cells from either PMEL or 2C mice were cultured on HA or TCP conditions. The cells were collected at designated time points. These cells were frozen down in liquid nitrogen for western blots, stored in TRIzol for mRNA detection, or PFA fixed for phospho-flow. For drugs, the final concentration of rapamycin (mTORC1 inhibitor), U-0126 (Erk1/2 inhibitor), blebbistatin, and anti-CD44 (KM201) were 0.1 μM, 10 μM, 100 μM, and 5 μg/mL, respectively. Half-volumes of T cell culture media was added every other day to keep cells in good condition. For western blot and rt-PCR, live cells that have been cultured for more than 24 hours were first purified using Ficoll-Paque followed by the procedure mentioned above.

### Western Blot

Frozen cells were lysed in a radioimmunoprecipitation assay (RIPA) buffer-based mixture containing proteinase inhibitor, PMSF (phenylmethane sulfonyl fluoride), sodium pyrophosphate, sodium fluoride, sodium orthovanadate, and β-glycerophosphate to inhibit phosphatases. Then, protein samples underwent standard western blot procedure with 1-2 hours of incubation in 5% milk, overnight incubation in primary antibodies (in 4% BSA), and 1 hour incubation in secondary antibodies. Films were imaged in a UVP BioSpectrum Imaging System, analyzed in UVP VisionWorks and quantified in ImageJ. The antibodies used are set forth below.

| Target | Cat# | Vendor |
|---|---|---|
| p-S6 (S240/244) | 2215 | Cell Signaling |
| p-Erk (Y202/204) | 4695 | Cell Signaling |
| Beta-actin | 4970 | Cell Signaling |
| p-S6K1 (T389) | 9234 | Cell Signaling |
| p-AKT (S473) | 3787 | Cell Signaling |

### RT-PCR

Cells were kept in TRIzol in −80° C. for storage. mRNA was purified using Zymo Quick-RNA MiniPrep Kit. Then, reaction mix was prepared based on a standard RT-PCR protocol. Probes were from TaqMan FAM/MGB probes with VIC/TAMRA Eukaryotic 18S rRNA as an endogenous control (ThermoFisher). Samples were run in quintuplicate in Applied Biosystems StepOnePlus Real-Time PCR system and analyzed using Excel.

### Phosphorylation Flow Cytometry

Cells were first stained with Live/Dead stain and then were fixed using BD Phosflow Fix Buffer I at room temperature for 10 minutes. After washing, cells were permeabilized using ice cold BD Phosflow Perm Buffer II for 30 minutes on ice. Samples were then stained with a solution of FACS wash buffer with 1:50 PE conjugated rat anti mouse CD8a, clone 53 6.7 (BD Pharmingen) and a 1:100 Rabbit anti Phospho S6 Ribosomal Protein (Ser235/236), clone D57.2.2E, or Rabbit IgG Isotype Control, clone DA1E (Cell Signaling Technology, Danvers, Mass.) for 45 minutes at room temperature. Samples were then washed with FACS wash buffer and stained with a solution of FACS wash buffer with 1:250 of Alexa Fluor 647-conjugated Goat S22 anti Rabbit IgG, polyclonal (ThermoFisher) for 45 minutes at room temperature. Samples were washed and resuspended with FACS wash buffer and read on a BD FACSCALIBUR™.

### T Cell Phenotype Assay

On day 7 of culture, the numbers of cells were counted using hemocytometer. After counting, less than 500,000 cells were collected and stained with a 1:100 PBS solution of APC-conjugated rat anti-mouse CD8a, clone 53-6.7 (BD Pharmingen), PE-conjugated rat anti-mouse CD62L, clone MEL-14 (BD Pharmingen), PerCP-conjugated rat anti-mouse CD44, clone IM7 (Biolegend), and 1:1000 of LIVE/DEAD® Fixable Green Dead Cell Stain (ThermoFisher) for 15 minutes at 4° C. Cells were then washed with FACS wash buffer to be read on BD FACSCALIBUR™ flow cytometer and analyzed using FLOWJO™ to measure the population of naïve T cells (CD62L-CD44+), effector T cells (CD62L+CD44-), and memory T cells (CD62L+CD44+). For human phenotype experiments, the same protocol was used, except the cells were instead stained with a 1:100 PBS solution of APC-conjugated anti-human CD45RA, Clone HI100 (Biolegend), PE-conjugated anti-human CD62L, clone DREG-56 (Biolegend), PerCP-conjugated anti-human CD8a, clone SK-1 (Biolegend), and 1:1000 of LIVE/DEAD® Fixable Green Dead Cell Stain for 15 minutes at 4° C. Cells were then washed with FACS wash buffer to be read on BD FACSCALIBUR™ flow cytometer and analyzed using FLOWJO™ to measure the population of naïve T cells (CD62L-CD45RA−), effector T cells (CD62L-CD45RA+), central memory T cells (CD62L+CD45RA−), and effector memory T cells (CD62L-CD45RA−).

For analysis of IL-7Ra and IL-15Ra expression on cells, on day 7 of culture the numbers of cells were counted using hemocytometer. After counting, samples were divided into four tubes (less than 500,000 cells per tube) and stained with a 1:100 PBS solution of APC-conjugated rat anti-mouse CD8a, clone 53-6.7 (BD Pharmingen), 1:1000 of LIVE/DEAD® Fixable Green Dead Cell Stain (ThermoFisher), and either PE-conjugated rat anti-mouse IL7Ra, clone A7R34 (Biolegend), or isotype control PE-conjugated Rat IgG2a, κ Isotype Ctrl, clone RTK2758 (Biolegened), or PE-conjugated rat anti-mouse IL15Ra, clone DNT15Ra (eBioscience), or isotype control PE-conjugated Rat IgG1, κ Isotype Ctrl, clone eBRG1 (eBioscience), for 15 minutes at 4° C. Cells were then washed with FACS wash buffer to be read on BD FACSCALIBUR™ flow cytometer and analyzed using FLOWJO™.

### T Cell Cytokine Functionality Assay

On day 7 of culture, approximately 500,000 CD8+ T cells were isolated from each condition and separated into restimulation and no-stimulation groups in 100 μL T cell culture media. To inhibit protein transport, 10 μL solution of 1:50 FITC anti-CD107a, 1:350 BD GolgiStop Protein Transport Inhibitor (BD Biosciences), and 1:350 BD GolgiPlug Protein Transport Inhibitor (BD Biosciences) in PBS was added to the samples. For the restimulation group, microparticle Dyanl-based aAPC were added at a 1:1 ratio. Both groups were incubated at 37° C. for 6 hours. After incubation, cells were washed and stained with 1:100 PBS solution of PerCP-conjugated anti-mouse CD8a, clone 53-6.7 (Biolegend) and 1:1000 of LIVE/DEAD® AmCyan Fixable Aqua Dead Cell Stain (ThermoFisher) at 4° C. for 30 minutes. Cells were then fixed and permeabilized with 100 μL BD Cytofix/Cytoperm Fixation and Permeabilization Solution (BD Biosciences) overnight. To analyze intracellular cytokines, cells were washed with 1×BD PERM/Wash buffer with 2% BSA the following day and stained with 1:100 solution of PE-conjugated rat anti-mouse IFN-γ, clone XMG1.2 (BD Pharmingen), APC-conjugated rat anti-mouse IL2, clone JES6-5H4 (BD Pharmingen), and PE-Cy7-conjugated rat anti-mouse TNFα, clone MP6-XT22 (Biolegend) in PERM/Wash buffer with 2% BSA at 4° C. for 1 hour. Stained cells were read on BD LSR II flow cytometer and analyzed by subtracting cytokine positive cells in the no-stimulation group from the re-stimulation group using FlowJo.

For antigen-specific cells a similar assay was used with the following modifications. Instead of a restimulation, cells were simply stained with 1 μg of either cognate or non-cognate biotinylated pMHC-Ig dimer for 1 hour at 4° C. After washing, samples were stained with a 1:350 ratio of PE-labeled streptavidin (BD Pharmingen). Then 10 μL solution of 1:50 FITC anti-CD107a, 1:350 BD GolgiStop Protein Transport Inhibitor (BD Biosciences), and 1:350 BD GolgiPlug Protein Transport Inhibitor (BD Biosciences) in PBS was added to the samples and incubated for 37° C. for 6 hours. Cells were then washed and stained with 1:100 PBS solution of PerCP-conjugated anti-mouse CD8a, clone 53-6.7 (Biolegend) and 1:1000 of LIVE/DEAD® AmCyan Fixable Aqua Dead Cell Stain (ThermoFisher) at 4° C. for 30 minutes. Cells were then fixed and permeabilized with 100 μL BD Cytofix/Cytoperm Fixation and Permeabilization Solution (BD Biosciences) overnight. Cells were then washed with 1×BD PERM/Wash buffer with 2% BSA and stained with 1:100 solution of APC-conjugated rat anti-mouse IFN-γ, clone XMG1.2 (BD Pharmingen) and PE-Cy7-conjugated rat anti-mouse TNFα, clone MP6-XT22 (Biolegend) in PERM/Wash buffer with 2% BSA at 4° C. for 1 hour. Stained cells were read on BD LSR II flow cytometer.

### Expansion of Rare Antigen-Specific T Cells

B6 CD8+ T cells were stimulated on aTM surfaces as described previously for 7 days. To detect antigen-specific CD8+ T cells, cells were stained with 1 μg of either cognate or non-cognate biotinylated pMHC-Ig dimer, with a 1:100 ratio of APC-conjugated rat anti-mouse CD8a, clone 53-6.7 (BD Pharmingen) in FACS wash buffer for 1 hour at 4° C. Samples were washed and then stained with a 1:350 ratio of PE-labeled streptavidin (BD Pharmingen) and a 1:1000 ratio of LIVE/DEAD™ Fixable Green Dead Cell Stain (ThermoFisher) in PBS for 15 minutes at 4° C. Cells were then washed and read on a BD FACSCALIBUR™. Percent antigen-specific cells were calculated by subtracting the percent gated in cognate stained CD8+ T cells from non-cognate stained CD8+ T cells. Number of antigen-specific cells was determined from multiplying the percent of antigen-specific cells by the number counted following cell harvest. Detection of antigen-specific human cells was done similarly, except instead of staining with biotynlated dimer, the antigen-specific cells were stained with purchased PE-labeled tetramer (MBL International, Woburn, Mass.) for 30 minutes at room temperature, then washed and stained with APC-conjugated anti-human CD8a, clone SK-1 (Biolegend), and 1:1000 of LIVE/DEAD® Fixable Green Dead Cell Stain for 15 minutes at 4° C.

For expansion of rare T cells from tumor-experienced mice, mice were injected with 2×10⁶ B16-SIY melanoma tumor cells expressing the SIY antigen and tumors were allowed to grow until on average were around 100 mm². CD8+ T cells were then harvested from the lymph nodes and spleens as previously described and expansion and detection were performed as previously described.

For analysis of IL7Ra of antigen-specific T cells, a similar process was used. Cells were stained with 1 μg of either cognate or non-cognate biotinylated pMHC-Ig dimer, with a 1:100 ratio of PerCP-conjugated rat anti-mouse CD8a, clone 53-6.7 (BD Pharmingen) in FACS wash buffer for 1 hour at 4° C. Samples were washed and then stained with a 1:350 ratio of PE-labeled streptavidin (BD Pharmingen), either APC-conjugated rat anti-mouse IL7Ra, clone A7R34 (Biolegend) or isotype control APC-conjugated Rat IgG2a, κ Isotype Ctrl, clone RTK2758 (Biolegend), and a 1:1000 ratio of LIVE/DEAD™ Fixable Green Dead Cell Stain (ThermoFisher) in PBS for 15 minutes at 4° C.

### Therapeutic Adoptive Transfer of T Cells

On day 0, B6 mice were injected with 2×10⁶ B16-SIY melanoma tumor cells expressing the SIY antigen. On day 1, CD8+ T cells were isolated from wildtype B6 mice and cultured for 7 days to produce stimulated T cells for adoptive transfer. On day 7, mice were given a central dose of 500 cGy, which induces transient lymphopenia similar standard approaches within adoptive immunotherapy85. On day 8, T cells cultured ex vivo were harvested and adoptively transferred intravenously in volumes of 100 pt. For every 3 mice receiving treatment, 1 B6 spleen was used for CD8+ T cell isolation and stimulation. Tumor sizes were measured using calipers and multiplying the longest measured length by the perpendicular direction of the tumor. Mice were sacrificed once tumors grew larger than 200 mm².

### Example 1

This example demonstrates a method of engineering an artificial T cell stimulating matrix (aTM).

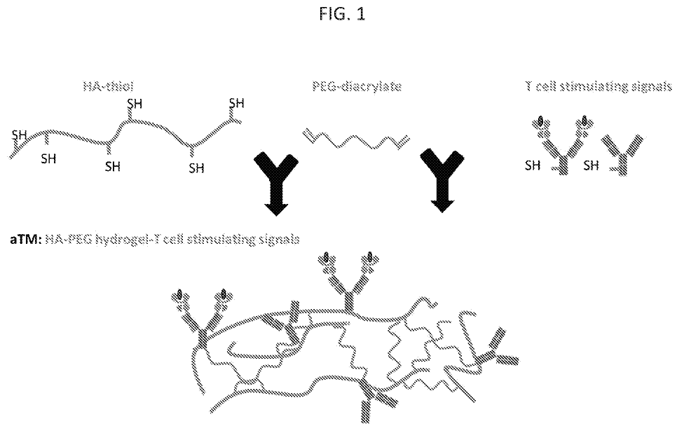

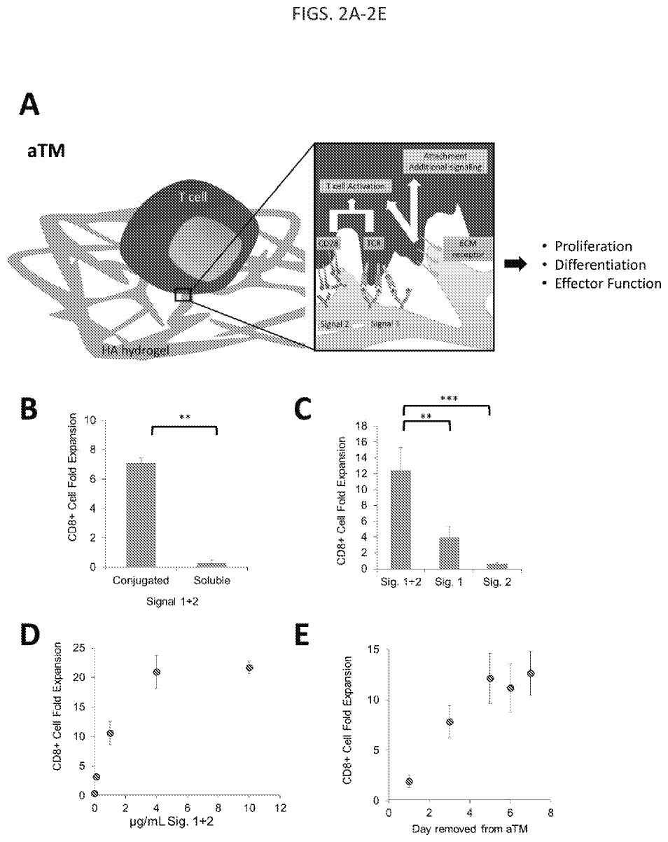

ECM hydrogels were formed by crosslinking thiolated hyaluronic acid (HA) with polyethylene glycol diacrylate (PEGDA) (FIG. 1). This material was engineered into an antigen-presenting material by conjugating the signals (Signal 1 and Signal 2) needed for T cell activation directly to the scaffold (FIG. 2A). Anti-CD3 and anti-CD28 antibodies were first used for polyclonal wildtype B6 murine CD8+ T cell expansion. This presents a unique approach to use the biophysical properties of hydrogels to influence the potency of a stimulatory environment, which were termed an artificial T cell stimulating matrix (aTM).

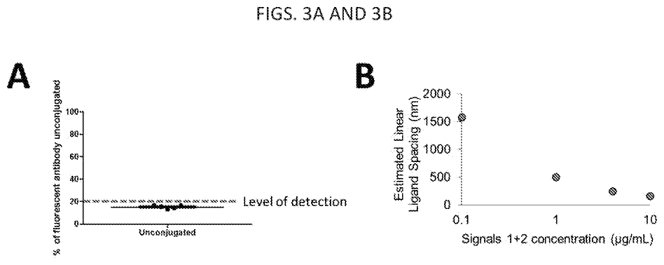

Post-conjugation, nearly all (at least 85%) of the stimulatory signals conjugated remained attached to the scaffold post-gelation (FIG. 3A). Direct conjugation of Signals 1 and 2 (i.e., aTM) mediated about 7-fold polyclonal T cell proliferation, whereas the same hydrogel substrate with soluble Signals 1 and 2 showed little proliferation (FIG. 2B). Substrates with only Signal 1 or 2 conjugated resulted in much lower T cell activation and proliferation (FIG. 2C).

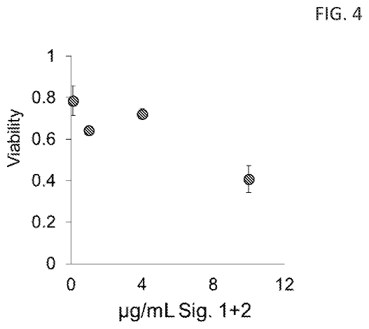

The density of T cell stimulating signals is an important parameter to control and optimize. As the concentration of stimulatory ligands was increased on the aTM, the amount of CD8+ T cell proliferation increased, though it plateaued at around 20-fold expansion when 4 μg/mL of Signals 1 and 2 was used (FIG. 1D). The surface density of the signals attached to the surface of the aTM was estimated for each concentration (FIG. 3B). The findings estimated that the spacing for ligands need to only be at least 500 nm apart (corresponding to 1 μg/mL), which is larger than previously reported values, potentially due to the fact that signals may not be evenly distributed across hydrogel surface and could be clustered on ECM polymers, or that compliant surfaces require less dense arrays of signal. Additionally, the viability of T cells decreased beyond 4 μg/mL of Signals 1 and 2 (FIG. 4), and thus less than 4 μg/mL or less were used for subsequent studies. Finally, CD8+ T cells required at least five days to be fully stimulated on the aTM surface, where suboptimal activation was observed when cells were removed from the hydrogels on days 1 and 3 (FIG. 2E), indicating a need for dynamic engagement of conjugated stimulatory molecules.

### Example 2

This example demonstrates the effects of hydrogel matrix stiffness on T cell stimulation.

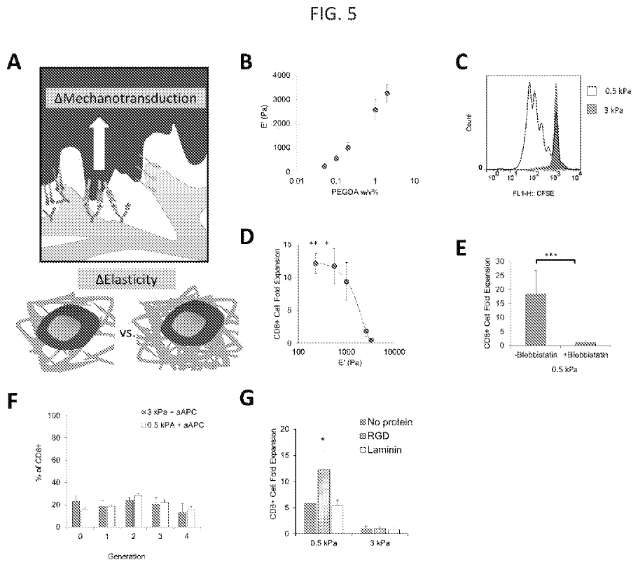

The matrix stiffness modulates cell function through mechanotransduction signaling mechanisms (Humphrey et al., Nat. Rev. Mol. Cell Biol., 15: 802 (2014); and Jaalouk, D. E. & Lammerding, J, Nat. Rev. Mol. Cell Biol., 10: 63 (2009). This factor may also contribute to CD8+ T cell stimulation on the aTM described in Example 1. Because the stimulatory signals are attached to the hydrogel ECM, this will likely affect the mechanical signaling through the TCR and CD28 molecules, as TCR activation has been shown to be a mechanosensitive process (see, e.g., Kim et al., J. Biol. Chem., 284: 31028-31037 (2009); Li et al. J. Immunol. ji_0900775 (2010); Das et al., Proc. Natl. Acad. Sci., 112: 1517-1522 (2015); Huse, M., Nat. Rev. Immunol. 17: 679 (2017); and Liu et al., Cell, 157: 357-368 (2014)) (FIG. 5A).

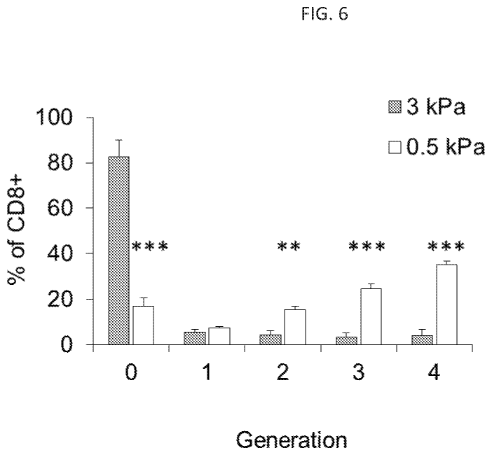

Secondary lymphoid tissue is a soft tissue and the stiffness has been reported to be between 0.1 to 2 kPa (Hirsch et al., Magn. Reson. Med., 71: 267-277 (2014); and Miyaji et al., Cancer Interdiscip. Int. J. Am. Cancer Soc., 80: 1920-1925 (1997)). To control the mechanical stiffness of the hydrogel within this range, the amount of crosslinker was altered to vary the elastic modulus from 0.2 to 3 kPa (FIG. 5B). A softer aTM (0.5 kPa) stimulated CD8+ T cell proliferation more effectively than the stiffer aTM (3 kPa) as determined by the dilution of the proliferation dye, carboxyfluorescein succinimidyl ester (CFSE) (FIG. 5C). Greater than 80% of the CD8+ T cells divided past the first and second generation when stimulated on the 0.5-kPa aTM, while the majority of T cells on 3-kPa aTM did not divide at all (FIG. 6). The spectrum of substrate stiffness was probed to determine the optimal range for T cell stimulation. aTMs with a stiffness below 1 kPa were more effective at stimulating CD8+ T cell expansion, where a dramatic decrease in T cell expansion was observed with aTM greater than 1 kPa (FIG. 5D).

Previous studies with CD4+ T cells found the most effective stimulation occurred near 100 kPa (close to the stiffness of bone) (Judokusumo et al., Biophys. J., 102: L5-L7 (2012); and O'Connor et al., J. Immunol., 189: 1330-1339 (2012)). In contrast, the optimum matrix stiffness for the disclosed hydrogel was nearly 100-fold less. Enhanced stimulation at lower stiffness could arise from the ECM-based matrix providing additional attachment points that promote the interaction between the cells and the hydrogel surface coated with stimulatory signals. Furthermore, previous studies attached stimulatory antibodies through either non-specific adsorption or through streptavidin and biotinylated antibodies, whereas the signals used herein are chemically attached to the matrix, which may contribute to more effective mechanotransduction at lower stiffness. Additionally, softer hydrogels which are more compliant may enable enhanced clustering of neighboring attached Signal 1 molecules on the hydrogel—shown to promote superior TCR signaling (Chaudhuri et al., Nat. Commun., 6: 6365 (2015); Hickey et al., Nano Lett., 17 (2017)). Finally, with softer hydrogel surfaces, the appropriate level of resistance that a T cell may observe at a cellular level is approached, where researchers have shown that the minimum adhesion strength to antigen presenting cells to be around 90 Pa (Kim et al., J. Biol. Chem. 284, 31028-31037 (2009); and Wülfing et al., PNAS, 95: 6302-6307 (1998)).

To evaluate the hypothesis of the role of mechanotransduction, additional experiments were performed which involved using myosin inhibitors, decoupling signaling components from the hydrogel, visualizing cellular attachment, and adding further cell-adhesive molecules. First, to the culture of a 0.5-kPa aTM, a blebbistatin was added, which is a myosin II inhibitor important in the role of T cell mechanotransduction (Ilani et al., Nat. Immunol., 10, 531 (2009); and O'Connor et al., J. Immunol., 189: 1330-1339 (2012). In the presence of the inhibitor, CD8+ T cell expansion was abolished even with the same amount of stimulatory ligand present (FIG. 5E).

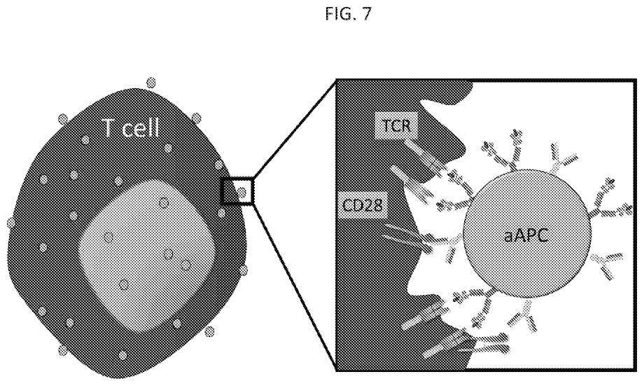

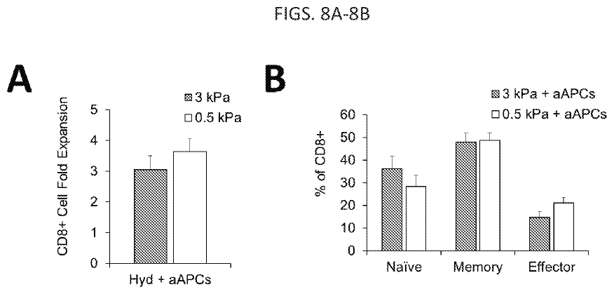

Second, the stimulatory agent was decoupled from the matrix and stimulated with cognate artificial antigen presenting cells (aAPC) on different stiffness of hydrogels. The nanoparticle aAPC contained both Signals necessary for CD8+ T cell activation—Signal 1: peptide loaded major histocompatibility complex (pMHC) and Signal 2: anti-CD28 co-stimulatory antibody (FIG. 7). There were no differences in the CFSE proliferation assay or in resultant cell phenotype between soft (0.5 kPa) and stiff (3 kPa) HA hydrogels (FIG. 5F and FIGS. 8A and 8B). This demonstrates that the mechanotransduction is independent of ECM-cell adhesion receptor interactions traditionally investigated, but instead dependent upon TCR signaling when stimulatory ligands are attached to HA hydrogels.

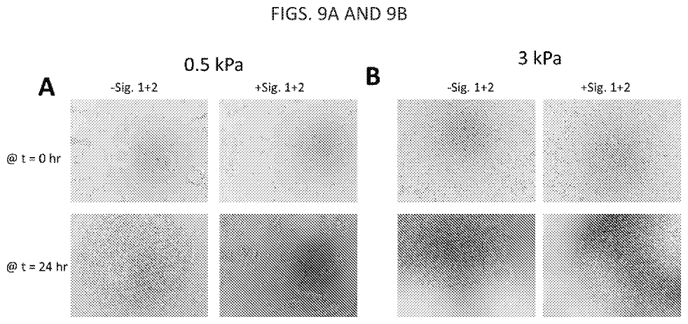

Third, the interaction of the T cells and the aTM hydrogels or HA hydrogels (without Signals 1 and 2) was visualized with light video microscopy (FIG. 9). After 24 hours, only CD8+ T cells remained attached to the soft (0.5 kPa) aTM hydrogel with both stimulatory signals conjugated. CD8+ T cells did not attach to stiff aTM (3 kPa) or soft (0.5 kPa) hydrogels without Signals 1 and 2 attached.

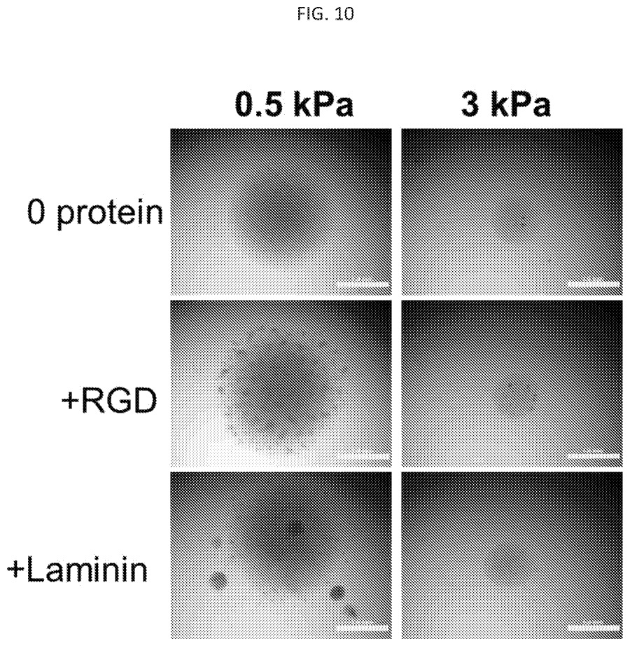

Fourth, adding cell-adhesive ligands has been shown to increase cell attachment to surfaces (Massia et al., Cell Biol., 114: 1089-1100 (1991)). Additional ECM-binding proteins were incorporated into the aTM scaffold such as laminin and cyclic RGD, a sequence derived from ECM-binding proteins to determine whether this might improve engagement and stimulation on stiff hydrogels. Even providing cell-adhesive ligands did not help stiff aTMs (3 kPa) stimulate antigen-specific PMEL CD8+ T cells, whereas RGD further increased T cell proliferation on soft (0.5 kPa) aTM surfaces resulting in effective expansion (FIG. 5G and FIG. 10).

Taken together, these data indicate that the role of mechanical stimulation is mediated through the TCR and the stimulatory ligands conjugated to the matrix, yet cannot be overcome with adding additional cell-attachment sequences.

### Example 3

This example describes the effects of the HA hydrogel on both T cell functionality and phenotype.

Beyond biophysical cues such as stiffness, the ECM can provide molecular signaling cues via cellular receptor activation. For T cells, CD44 has primarily been utilized as a marker for cellular phenotype and not examined as a co-stimulatory molecule (Sanders et al., J. Immunol., 140: 1401-1407 (1988); Graham et al., Eur. J. Immunol., 37: 925-934 (2007); Budd et al., J. Immunol., 138: 1009-1013 (1987); and Budd et al., J. Immunol., 138: 3120-3129 (1987)). To investigate how the HA hydrogel contributes to T cell activation and signaling, attachment of T cell stimulatory signals to the surface was decoupled by utilizing aAPC for T cell stimulation. In this manner, differences in T cell signaling directly due to the HA hydrogel could be mechanistically studied.

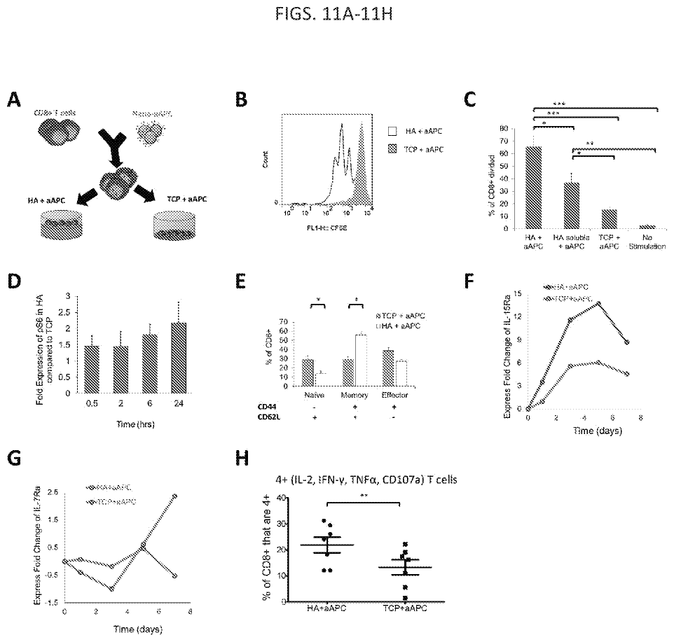

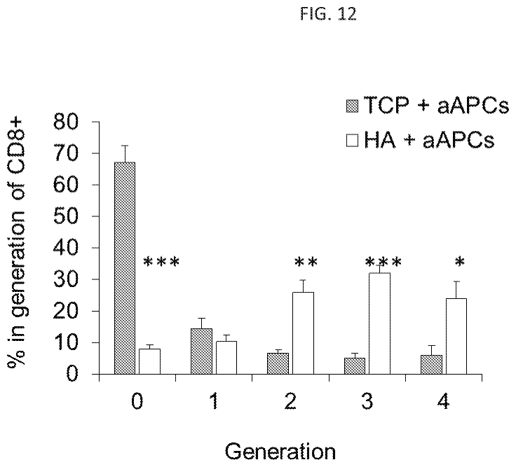

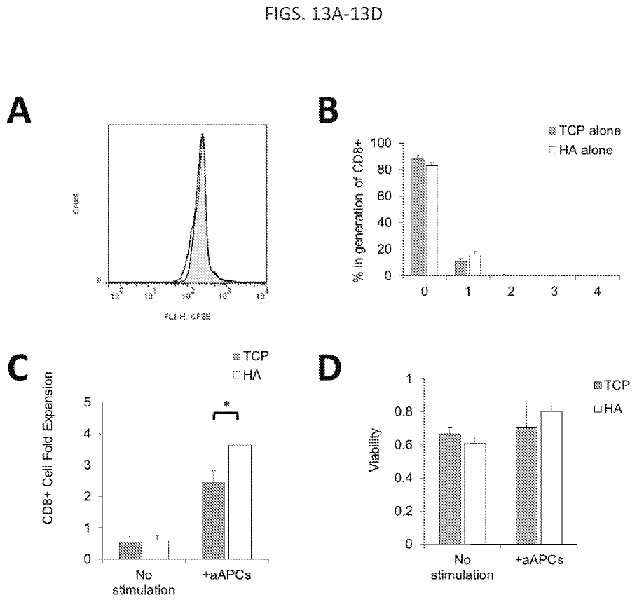

To examine the influence of stimulatory environment, transgenic PMEL CD8+ T cells and cognate aAPC were co-incubated and then either plated onto ECM-mimic hydrogels (HA) or the traditional tissue culture plate (TCP) wells (FIG. 11A). Interestingly, CD8+ T cells that were cultured on HA hydrogel surfaces demonstrated much higher antigen-specific T cell proliferation as indicated by CFSE dilution after three days of culture (FIG. 11B). In fact, there were significantly more T cells that reached the second, third, and fourth generations when compared to the T cells cultured on traditional tissue culture plates, where the majority of the cells had not yet divided (FIG. 12). There was no inherent signaling or activation without stimulatory aAPC with no effects on cell viability on the HA surface (FIGS. 13A-13D). Moreover, including soluble HA also increased the percent of CD8+ T cells dividing (˜35%) as compared to the tissue culture plate without hydrogel (˜15%), but not as much as when HA was crosslinked into a hydrogel (˜65%) (FIG. 11C). Therefore, the benefit of the HA to early CD8+ T cell proliferation is partially mediated through direct interaction with crosslinked HA in combination with TCR signaling.

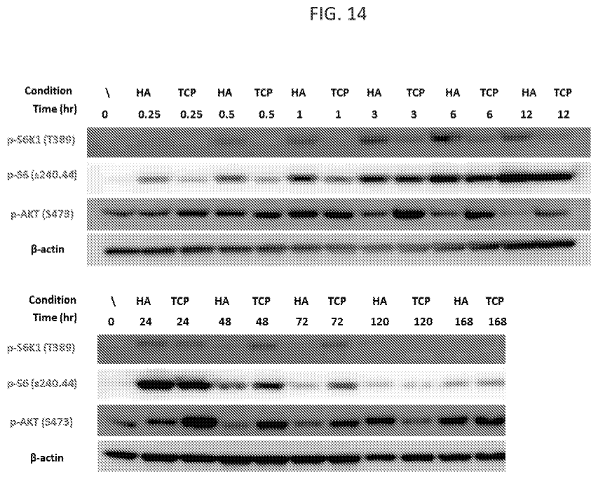

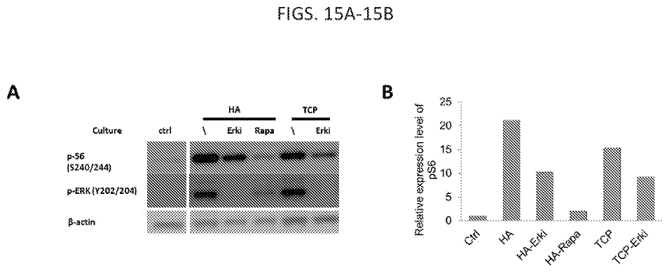

The role of HA in signaling and inducing greater early expansion of CD8+ T cells in the hydrogel condition was investigated. Exploring key signaling pathways related to T cell activation and proliferation, a significant increase in expression of p-S6K1 and p-S6 (FIG. 11D, FIG. 14) was observed, consistent with upregulation of mTORC1 (mammalian target of rapamycin complex 1), and a downregulation of p-AKT (indicative of mTORC2) under the HA culture condition compared to the TCP condition (FIG. 14). Furthermore, CD44-signaling has been shown to trigger Ras-Erk signaling in other cell types (Ponta et al., Nat. Rev. Mol. Cell Biol., 4: 33-45 (2003)), and Ras-Erk and PI3K-mTOR pathways have been shown to crosstalk and compensate for each other in T cells (Mendoza et al., Trends Biochem. Sci., 36: 320-328 (2011)). A significant amount of p-S6 signal may come from CD44-induced Ras-Erk signaling (FIGS. 15A and 15B).

Taken together, these results demonstrate that HA-CD44 signaling induces cell priming and expansion through crosstalk between Ras-Erk and PI3K-mTOR pathways, where mTOR is an important integrator of immune cues for robust T cell activation and phenotype skewing (Pollizi et al, J. Clin. Invest., 125: 2090-2108 (2015)).

### Example 4

This example demonstrates that the aTM hydrogel matrix described herein stimulates and polarizes human CD8+ T cells.

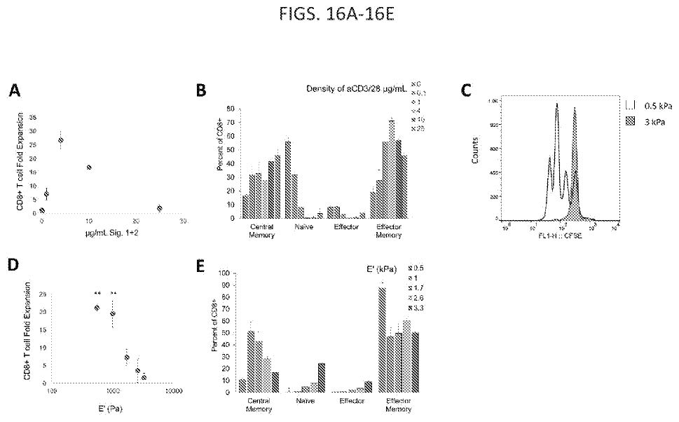

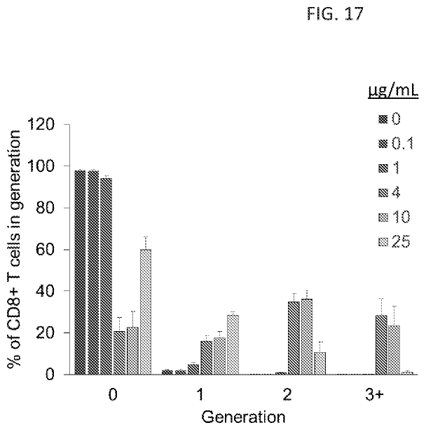

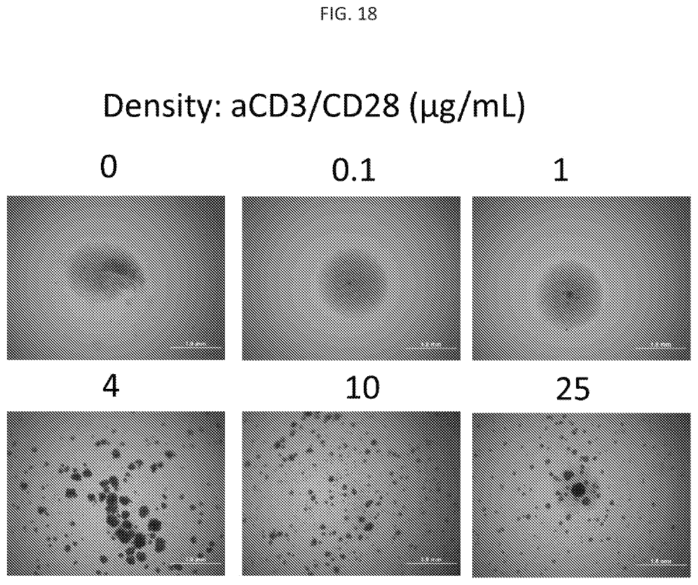

To show that the aTM described above also is capable of stimulating human CD8+ T cells, anti-human CD3 (Signal 1) and anti-human CD28 (Signal 2) were attached to the HA hydrogels. A similar increase in the fold expansion was observed when increasing the density of Signal 1 and 2 to 4 μg/mL (˜25 fold CD8+ T cell expansion in 1 week) on 0.5-kPa aTM; however, beyond this value, the fold proliferation of the cells dramatically decreased where little to no expansion was detected in the 25 μg/mL condition (FIG. 16A), with minimal CFSE dilution (FIG. 17). Nevertheless, phenotypic studies revealed that the cells were still proliferating at this dose, albeit at lower frequency (FIG. 16B). Interestingly, this indicates control over phenotype independent of cell proliferation. This high antibody density may provide too strong of an interaction and inhibit CD8+ T cell migration on the hydrogel, preventing beneficial intercellular interactions, as macroscopically more punctate, smaller cell clusters were observed in these hydrogels than in the 4 μg/mL (FIG. 18).

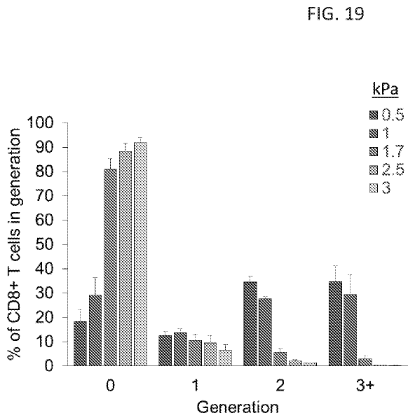

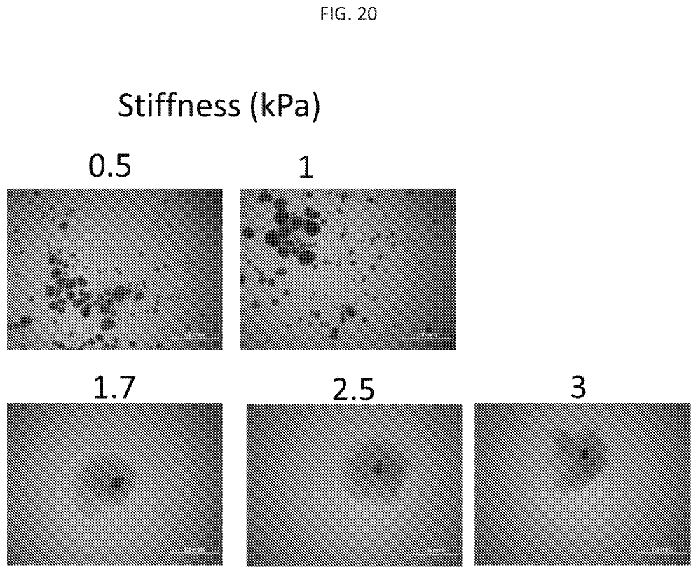

Similar matrix stiffness-dependent effects were observed where more effective stimulation (>20 fold expansion in 1 week) was observed on aTM hydrogels with an elastic modulus less than 1 kPa (FIGS. 16C and 16D; FIG. 19, FIG. 20). By changing the stiffness of the aTM, differences in phenotype were observed even within conditions that have similar fold expansions (FIG. 16E). For example, the 0.5 kPa and 1 kPa aTMs both provided nearly 20-fold expansion, but the 1 kPa aTM generated a more balanced ratio of central memory to effector memory CD8+ T cells than the 0.5 kPa aTM.

The results of this example demonstrate the creation of an aTM that stimulates and polarizes human CD8+ T cells for potential ACT therapy.

### Example 5

This example demonstrates that aTM-stimulated endogenous, antigen-specific T cells inhibit established tumor growth.

A main goal of ACT is to be able expand rare (e.g., frequency of 1 in 10⁵ to 10⁶ CD8+ T cells), antigen-specific CD8+ T cells to high numbers that are functional. Because of the difficulty in obtaining and activating these cells, most studies investigate the antigen-specific activation of T cells from transgenic mice or the non-specific activation of endogenous T cells. This limits clinical relevance because of lack of translatability and the monoclonality of these T cells. Thus, an optimized version of the aTM in the setting of activating rare antigen-specific CD8+ T cells was investigated. To this end, instead of using non-specific Signal 1 (anti-CD3), antigen-specific Signal 1 (pMHC: Kb-SIY) was conjugated to aTM with co-stimulatory anti-CD28, and stimulating conditions similar to those used in Example 3 were compared with T cells mixed with aAPC cultured on HA hydrogel or TCP surface.

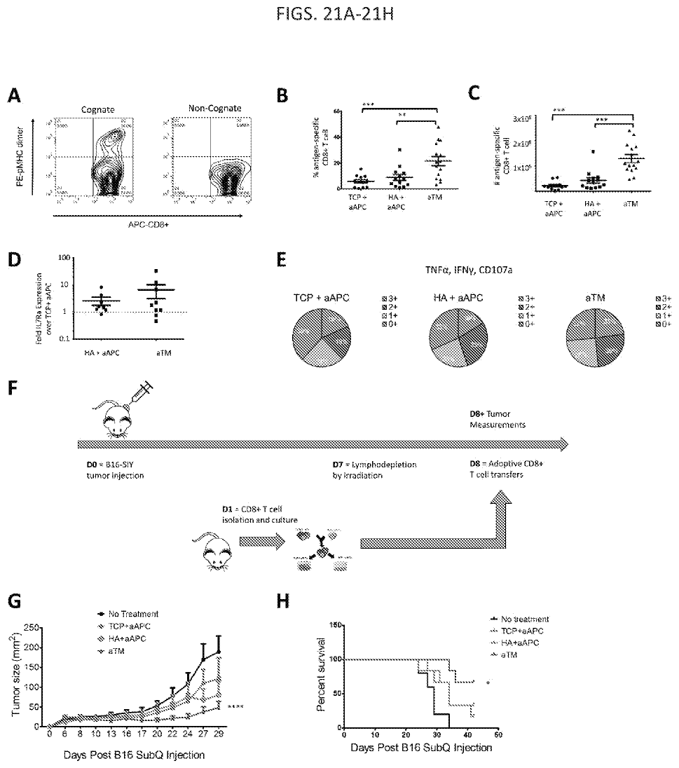

After seven days of stimulation, the antigen-specificity of the cultures was determined, and it was found that up to 40% of the CD8+ T cells were antigen-specific from aTM cultures (FIG. 21A). Indeed, more than double the percentage of antigen-specific cells and more than quadruple the total number of antigen-specific cells expanded on aTM was observed (FIGS. 21B and 21C). This highlights the importance of studying endogenous T cell activation. Such a drastic increase in cell number, even between the aTM and HA+aAPC groups, where the only difference was the location of the stimulatory signals, was surprising. Therefore, the biophysical aspects of engaging the TCRs and CD28 from the HA hydrogel surface represent important progress in activating antigen-specific T cells effectively.

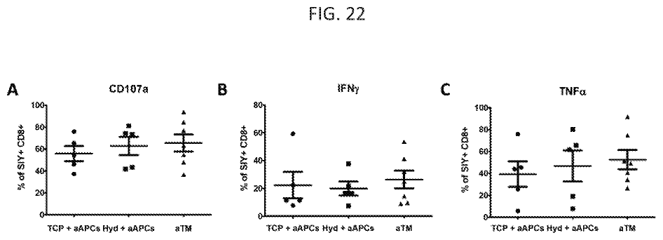

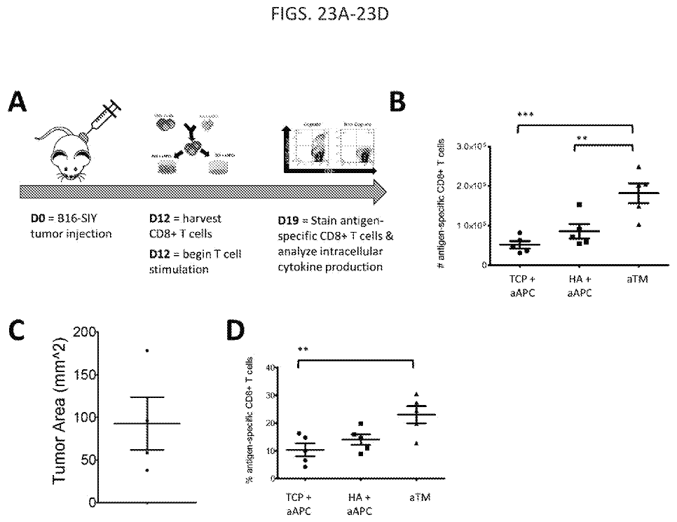

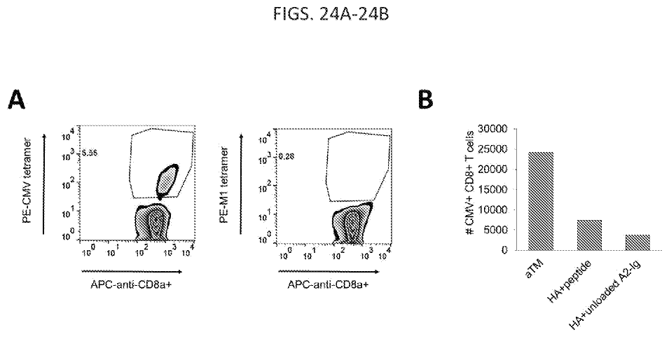

Since differences in the IL7Ra expression and functionality of the CD8+ T cells cultured on the HA hydrogel was observed, antigen-specific cells after seven days of stimulation were probed for these markers. Again, there was an increase in both the IL7Ra (FIG. 21D) and the functionality associated with an increase in the percent of SIY+ T cells that were positive for multiple cytokines and degranulation markers (FIG. 21E, FIG. 22). These results are significant, as they show that aTM is capable of generating higher numbers of functional antigen-specific CD8+ T cells. These findings were consistent with CD8+ T cells isolated from mice with established tumors (FIG. 23), and demonstrated that human antigen-specific T cells could be expanded with aTM specific for CMV+CD8+ human T cells (FIG. 24).

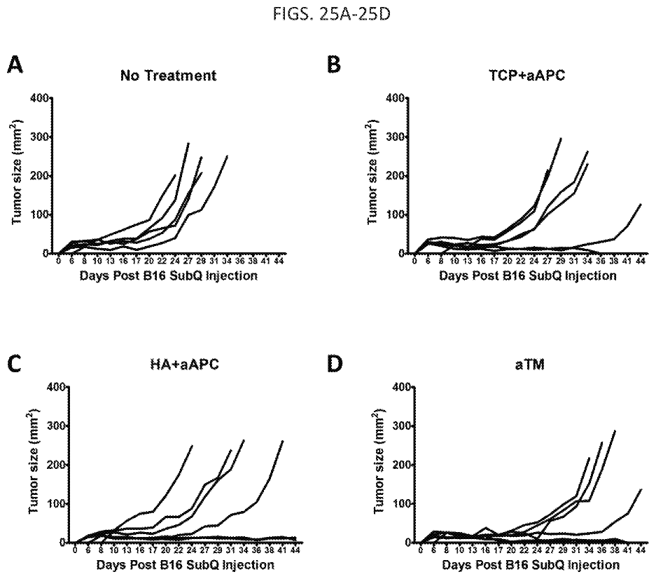

Finally, the in vivo activity of aTM-stimulated and expanded T cells were tested in an ACT model where T cells were transferred into mice with established B16-SIY melanoma tumors (FIG. 21F). T cells stimulated by aTM significantly reduced tumor growth as compared to T cells on other surfaces and no treatment controls. Even on day 29, tumors in the group receiving aTM-stimulated T cells were stable below 50 mm² (FIG. 21G, FIGS. 25A-25D). This treatment also resulted in improved survival rate. By day 40, none among the no treatment group, 16% of TCP+aAPC treated mice, and 33% of HA+aAPC treated group survived. In contrast, 66% of mice survived after receiving aTM-stimulated T cells at the same dose (FIG. 21H). Thus, T cells stimulated on the aTM were most effective in halting tumor growth.

### Example 6

This example describes the generation of an artificial lymph node (aLN) with the hyaluronic acid (HA) hydrogel described herein and its ability to activate adoptively transferred antigen-specific CD8+ T cells.

Artificial lymph nodes (aLNs) were synthesized by mixing 0.8 w/v % thiolated HA with either 0.1 or 2 w/v % of the crosslinker PEG-diacrylate (PEGDA), which was pre-mixed with 5 μg/mL thiolated anti-CD3 and anti-CD28 antibodies, and either no protein, 100 μM cyclic RGD, or 20 μg/mL laminin was added. Isolated murine CD8+ T cells were added to the surface of the aLNs. On day 7 viable cells were harvested, counted, and stained for phenotypic markers for flow cytometry (CD8, CD44, CD62L). For in vivo experiments, B6 mice received subcutaneous injection of aLN treatment (no protein, 570 Pa, with antigen-specific stimulation from KbSIY for 2C CD8+ T cells) or PBS. 2C CD8+ T cells were isolated, labeled with CellTrace CFSE dye and injected retro-orbitally on the same day. On days 3 and 6, blood samples were taken for CFSE division analysis via flow cytometry.

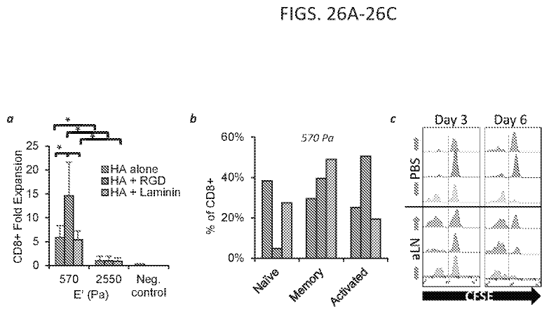

CD8+ T cells cultured on the softer (570 Pa) aLN surfaces induced nearly 10 fold more CD8+ T cell proliferation than those of stiffer counterparts (2550 Pa), regardless of protein attachment (FIG. 26A). Cyclic RGD attachment nearly doubled total expansion compared to no protein and laminin on softer aLNs (FIG. 26B). The stiffness may alter force generation on T cells via aCD3/28 signaling, and cyclic RGD may enhance this force sensation allowing further contact with the aLN. Alternatively, RGD and laminin may also provide a third signal to the T cells, supported by observed differences T cell phenotype, where percent memory and activated are increased by RGD and laminin, respectively (FIG. 26B). Finally, the aLN demonstrated in vivo efficacy by observed decrease of CFSE dilution in antigen-specific, adoptively transferred CD8+ T cells at days 3 and 6 (FIG. 26C).

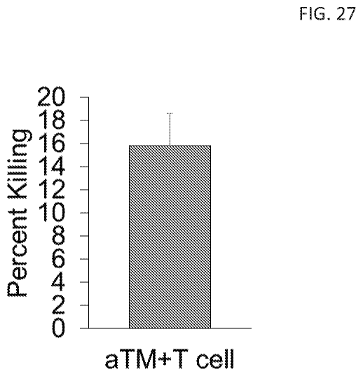

In vivo function of the aLN was further investigated by performing an adoptive transfer of transgenic 2C CD8+ T cells with an aLN comprising the HA hydrogel described herein in microparticle form (KbSIY, anti-CD28, and an anti-IL-2 antibody conjugated to IL-2). The microparticles were injected s.c. and allowed to stimulate transferred naïve T cells for 5 days before CFSE labeled target cells were injected for an in vivo killing assay compared to no treatment controls. Nearly 15% killing from the aLN and T cell treated mice was observed, indicating function of the adoptively transferred T cells (FIG. 27).

This example demonstrates the design of an aLN with mechanical properties similar to secondary lymphoid tissue, which has been reported to be around 0.1-2 kPa (Hirsch et al., supra). Stiffness and protein attachment impacted final CD8+ T cell stimulation and phenotype. The created aLNs also demonstrated in vivo efficacy in an adoptive transfer mouse model.

### Example 7

This example describes the generation of an artificial lymph node (aLN) using a hydrogel comprising a polymeric nanofiber.

To mimic the unique structure of the lymph node, which contains long collagen-based reticular fiber networks, polymeric nanofibers were incorporated into the ECM-based aLNs described above to generate more high-quality CD8+ T cells for cancer immunotherapy.

Poly-caprolactone (PCL) nanofibers were prepared by electrospinning and attaching varying amounts of stimulatory signal to the surface through EDC/NHS chemistry. Transgenic 2C CD8+ T cells were stimulated for 7 days and counted to measure fold expansion. Composite aLN were prepared similarly to the hydrogel aLN described in Example 6, but PCL nanofibers modified with maleimide groups were added at 0.5 w/v % to the hydrogel aLN. To evaluate cell functionality, intracellular cytokine staining (ICS) was performed on harvested cells to measure INF-γ, TNFα, IL-2, and CD107a+ T-cells.

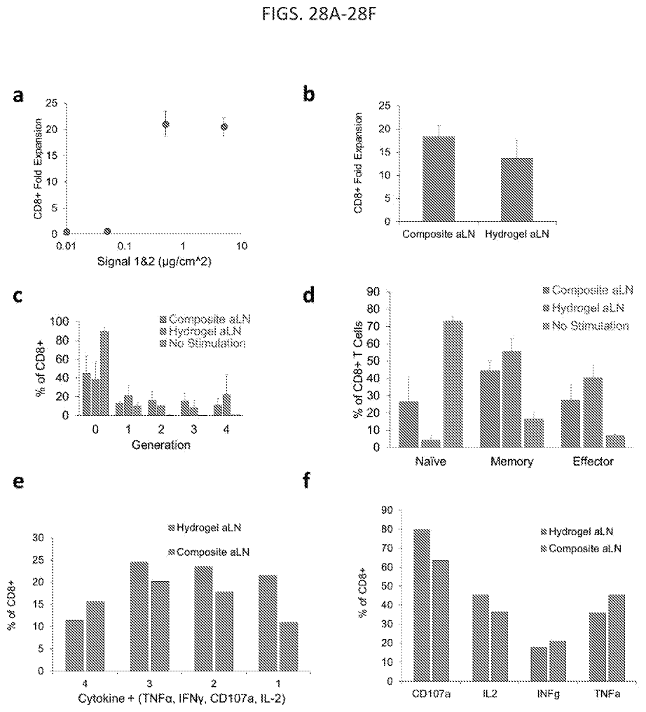

PCL nanofibers act as aAPCs and activate antigen-specific T cells with 0.5 μg/cm2 of stimulatory signal or greater (FIG. 28A). Composite aLNs with PCL nanofibers induced similar levels of expansion by day 7 fold proliferation (FIG. 28B) and by CFSE (FIG. 28C), with similar phenotype (FIG. 28D) and functional profiles (FIGS. 28E and 28F) compared to hydrogel aLNs.

aLNs containing polymeric nanofibers provide an environment that readily produces high numbers of functional antigen-specific T-cells. The aLNs not only increase the number of antigen specific CD8+ T-cells, but also T-cell functionality. Furthermore, the addition of polycaprolactone (PCL) nanofibers in an ECM matrix produces aLNs that more closely mimic lymph node physiology in structure, stiffness, and porosity that further enhance T-cell stimulation. Beyond demonstrating the importance of biomimetic biomaterial design, these data have implications for use of these cells in adoptive immunotherapy and direct use of these materials as in vivo therapies.

### Example 8

This example describes a method of generating microgel particles (MPs) out of the aTM hydrogels described herein.

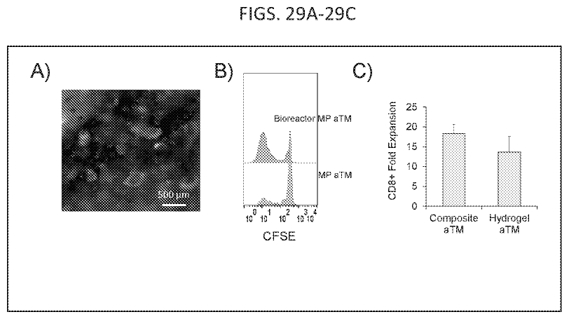

The lymph node is one of the most cellularly dense organs, and T cells scan DCs in every axis. To mimic this environment, a method was developed to generate microgel particles (MPs) out of the aTM hydrogels described herein by pushing them through stainless steel meshes with a defined pore size of 150 μm (FIG. 29A). The microgel form of the aTM stimulated CD8+ T cells effectively as shown by the CFSE dilution data but required direct contact between T cells and MPs by culture in a bioreactor, since MPs will float in normal media concentrations (FIG. 29B). In addition, nanofiber composite hydrogels have been created which have shown excellent immune cell infiltration and tissue integration in vivo by attaching milled, electrospun nanofibers to the ECM hydrogels. A composite aTM has been generated which stimulates T cells similarly to the ECM hydrogel aTM.

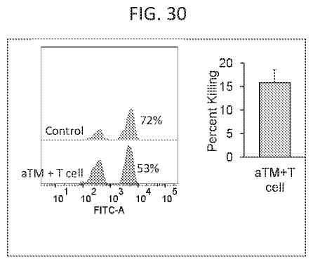

As a preliminary experiment to show the use of the hydrogel formulation for in vivo T cell activation, naïve 2C transgenic CD8+ T cells and KbSIY and anti-CD28-incorporated aTM microgels were injected subcutaneously into mice. After 7 days, a control (CFSE low) and target (CFSE high) population loaded with an irrelevant peptide and target peptide SIY, respectively, were differentially labeled. These cells were adoptively transferred into control and treated mice. After one day, lymphoid organs were removed killing of target population was observed, indicating successful in vivo stimulation (FIG. 30).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising a hydrogel having a shear modulus of about 20 Pa to about 1600 Pa conjugated with a first molecule that provides a first T cell activating signal and a second molecule that provides a second T cell activating signal.

2. The composition of claim 1, wherein the first molecule is an antigen presenting complex or an anti-CD3 antibody.

3. The composition of claim 2, wherein the antigen presenting complex comprises a peptide antigen in the context of MHC class I or II molecular complex.

4. The composition of claim 3, wherein the peptide antigen is a cancer-specific antigen, a cancer neoantigen, an autoantigen, or an infectious agent antigen.

5. The composition of any one of claims 1-4, wherein the second molecule is a T cell co-stimulatory molecule.

6. The composition of claim 5, wherein the co-stimulatory molecule specifically binds to CD28, CD80 (B7-1), CD86 (B7-2), B7-H3, 4-1BB, 4-1BBL, CD27, CD30, CD 134 (OX-40L), B7h (B7RP-1), CD40, or LIGHT.

7. The composition of any one of claims 1-6, wherein the first molecule is an anti-CD3 antibody and the second molecule is an anti-CD28 antibody.

8. The composition of claim 7, wherein the concentration of each of the anti-CD3 antibody and the anti-CD28 antibody is about 0.1 μg/mL to about 20 μg/mL.

9. The composition of claims 1-8, wherein the hydrogel comprises a crosslinked hyaluronic acid network.

10. The composition of claim 9, wherein the hydrogel comprises an acrylated hyaluronic acid crosslinked with polyethylene glycol diacrylate (PEGDA).

11. The composition of any one of claims 1-10, wherein the hydrogel further comprises a cell adhesion peptide.

12. The composition of claim 11, wherein the cell adhesion peptide is selected from one or more from the group including a RGD peptide, cyclic RGD peptide, YIGSR peptide, and IKVAV peptide.

13. The composition of any one of claims 1-12, which further comprises a cell adhesion protein selected from a group including laminin, collagen, fibronectin, fibrinogen, and fibrin.

14. The composition of any one of claims 1-13, which further comprises one or more nanofibers.

15. The composition of claim 14, wherein the one or more nanofibers are selected from a group including poly(ε-caprolactone), polylactide, polyglycolide, poly(lactide-co-glycolic acid) nanofibers.

16. A method of activating T cells comprising:
(a) contacting one or more T cells with the composition of any one of claims 1-15 and
(b) culturing the one or more T cells and the composition under conditions whereby the one or more T cells proliferate and differentiate.

17. The method of claim 16, wherein the T cells are CD8+ T cells and/or CD4+ T cells.

18. The method of claim 17, wherein the CD4+ T cells are selected from the group consisting of TH1, TH2, and Tregs CD4+ T cells.

19. The method of claim 16, wherein the one or more T cells are obtained from one or more of human PBMC, human tumor infiltrating lymphocytes, or bone marrow cells.

20. The method of any of claims 16-19, wherein the T cells are activated and cultured in vitro.

21. The method of any of claims 16-19, wherein the T cells are activated in vivo.

22. The method of claim 21, wherein the T cells are human PBMC and are activated and cultured in vitro.

23. A method for activating one or more T cells in vivo, the method comprising administering to a subject a composition of any one of claims 1-15.

24. The method of claim 23, wherein the administering of the composition to the subject treats an antigen-specific or an immunostimulatory disease or condition.

25. The method of claim 24, wherein the antigen-specific disease or condition is a cancer or an infectious disease.

26. The method of claim 24, wherein the immunostimulatory disease or condition is related to Human Immunodeficiency Virus (HIV) or one or more other immune targeting viruses.

27. The method of claim 23, wherein the administering of the compound is subcutaneously, intraperitoneally, or intratumorally.

28. A method of killing cancer cells in a subject, which method comprises:
(a) contacting one or more T cells isolated from the subject with the composition of any one of claims 1-15;
(b) culturing the one or more T cells under conditions whereby the one or more T cells proliferate and differentiate; and
(c) administering the activated T cells to the subject, whereby a cancer-specific immune response is induced in the subject and the cancer cells are killed.

29. A method of killing cancer cells in a subject, which method comprises administering the composition of any one of claims 1-15 to the subject, whereby one or more T cells in the subject proliferate and differentiate into effector T cells and/or memory T cells specific for the cancer and the cancer cells are killed.

30. The method of claim 28 or claim 29, wherein the subject is a human.

31. The method of claim 28 or claim 29, wherein the cancer is melanoma, leukemia, multiple myeloma, prostate cancer, breast cancer, lung cancer, or colorectal cancer.

32. The method of any one of claims 23-32, wherein the composition is administered in combination with T cells isolated from the subject or a donor other than the subject.


SEQUENCE LISTING (1 sequence)

SEQ ID NO: 1 - protein, 9 aa, Artificial sequence
  Synthetic cyclic RGD peptide
CCRRGDWLC